SEQUENCE LISTING (140 sequences)

SEQ ID NO: 1 - protein, 233 aa, Homo sapiens
  source 1..233: note = Full length TNF-alpha
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 2 - protein, 157 aa, Homo sapiens [sequence omitted]

SEQ ID NO: 3 - protein, 146 aa, Homo sapiens
  source 1..146: note = THD Domain with TNFR2 Agonist Sequences
PVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 4 - protein, 11 aa, Homo sapiens
  source 1..11: note = Sequence from the ADAM17 cleavage site in the stalk region to the C-terminus of the stalk region
VRSSSRTPSDK

SEQ ID NO: 5 - protein, 232 aa, Homo sapiens
  source 1..232: note = Human IgG1 Fc
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 232 aa, Homo sapiens
  source 1..232: note = Human IgG1 Fc with FcyR and C1q knockout
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 237 aa, Homo sapiens
  source 1..237: note = Human IgG1 Fc with N-terminal linker
GGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 237 aa, Homo sapiens
  source 1..237: note = Human IgG1 Fc with FcyR and C1q knockout and linker
GGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 255 aa, Homo sapiens
  VARIANT 235..255: note = GGGGS repeats may be deleted
  source 1..255: note = Human IgG1 Fc with C-terminal linker
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGS

SEQ ID NO: 10 - protein, 255 aa, Homo sapiens
  VARIANT 235..255: note = GGGGS repeats may be deleted
  source 1..255: note = Human IgG1 Fc with FcyR and C1q knockout and C-terminal linker
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGS

SEQ ID NO: 11 - protein, 229 aa, Homo sapiens
  source 1..229: note = Human IgG4 Fc
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 229 aa, Homo sapiens
  source 1..229: note = Human IgG4 Fc with Ser to Pro mutation
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 234 aa, Homo sapiens
  source 1..234: note = Human IgG4 Fc with N-terminal linker
GGGGSESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 234 aa, Homo sapiens
  source 1..234: note = Human IgG4 Fc with Ser to Pro Mutation and N-terminal linker
GGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 252 aa, Homo sapiens
  VARIANT 233..252: note = GGGGS repeats may be deleted
  source 1..252: note = Human IgG4 Fc with C-terminal linker
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGGGGSGGGGSGGGGS

SEQ ID NO: 16 - protein, 252 aa, Homo sapiens
  VARIANT 232..252: note = GGGGS repeats may be deleted
  source 1..252: note = Human IgG4 Fc with Ser to Pro Mutation and C-terminal linker
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGGGGSGGGGSGGGGS

SEQ ID NO: 17 - protein, 228 aa, Homo sapiens
  source 1..228: note = Human IgG2 Fc
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 18 - protein, 228 aa, Homo sapiens
  source 1..228: note = Human IgG2 Fc with C1q knockout
ERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 19 - protein, 233 aa, Homo sapiens
  source 1..233: note = Human IgG2 Fc with N-terminal linker
GGGGSERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 20 - protein, 233 aa, Homo sapiens
  source 1..233: note = Human IgG2 Fc with C1q knockout and N-terminal linker
GGGGSERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 21 - protein, 251 aa, Homo sapiens
  VARIANT 232..251: note = GGGGS repeats may be deleted
  source 1..251: note = Human IgG2 Fc with C-terminal linker
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGSGGGGSGGGGSGGGGSGGGGS

SEQ ID NO: 22 - protein, 251 aa, Homo sapiens
  REGION 232..251: note = GGGGS repeats may be deleted
  source 1..251: note = Human IgG2 Fc with C1q knockout and C-terminal linker
ERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGSGGGGSGGGGSGGGGSGGGGS

SEQ ID NO: 23 - protein, 106 aa, synthetic construct
  source 1..106: note = EHD2 dimerization domain
DFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSN

SEQ ID NO: 24 - protein, 111 aa, synthetic construct
  source 1..111: note = MHD2 dimerization domain
AELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPD

SEQ ID NO: 25 - protein, 5 aa, synthetic construct
  source 1..5: note = linker
GGGGS

SEQ ID NO: 26 - protein, 31 aa, synthetic construct
  source 1..31: note = TNF-a stalk region
GPQREEFPRDLSLISPLAQAVRSSSRTPSDK

SEQ ID NO: 27 - protein, 10 aa, synthetic construct
  source 1..10: note = linker
GGGGSGGGGS

SEQ ID NO: 28 - protein, 8 aa, synthetic construct
  source 1..8: note = stalk based linker
SSRTPSDK

SEQ ID NO: 29 - protein, 16 aa, synthetic construct
  source 1..16: note = G/S Stalk based linker
GGGGSVRSSSRTPSDK

SEQ ID NO: 30 - protein, 13 aa, synthetic construct
  source 1..13: note = G/S Stalk based linker
GGGGSSSRTPSDK

SEQ ID NO: 31 - protein, 700 aa, synthetic construct
  source 1..700: note = IgG1 Fc with mutations;GGGGS2; THDR2; stalk linker;THDR2; stalk linker; THDR2
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 32 - protein, 710 aa, synthetic construct
  source 1..710: note = IgG1 Fc with mutations; G/S stalk linker; THDR2; G/Sshort stalk linker; THDR2; G/S short stalk linker; THDR2
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 33 - protein, 157 aa, synthetic construct
  source 1..157: note = Soluble TNF-a sequence with TNFR2 agonist mutations
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 34 - protein, 701 aa, synthetic construct
  source 1..701: note = scTNFR2 agonist with C-terminal IgG1 with mutations.This is soluble TNF-a sequence, which includes VRSSSRTPSDK atN-terminus prior to THD domain.
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 35 - protein, 706 aa, synthetic construct
  source 1..706: note = scTNFR2 agonist with C-terminal IgG1 with mutations.This is soluble TNF-a sequence, which includes VRSSSRTPSDK atN-terminus prior to THD domain and GGGGS prior to IgG1.
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 36 - DNA, 2100 bp, synthetic construct
  source 1..2100: mol_type = other DNA; note = nucleic acid encoding SEQ ID NO:31 optimized for Musmusculus expression by www.jcat.de
gagcccaagagcagcgacaagacccacacctgccccccctgccccgcccccgaggccgccggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgccagcatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcggcggcggcagcggcggcggcggcagccccgtggcccacgtggtggccaacccccaggccgagggccagctgcagtggctgaacaggagggccaacgccctgctggccaacggcgtggagctgagggacaaccagctggtggtgcccagcgagggcctgtacctgatctacagccaggtgctgttcaagggccagggctgccccagcacccacgtgctgctgacccacaccatcagcaggatcgccgtgagctaccagaccaaggtgaacctgctgagcgccatcaagagcccctgccagagggagacccccgagggcgccgaggccaagccctggtacgagcccatctacctgggcggcgtgttccagctggagaagggcgacaggctgagcgccgagatcaacaggcccgactacctgaacttcagggagagcggccaggtgtacttcggcatcatcgccctggtgaggagcagcagcaggacccccagcgacaagcccgtggcccacgtggtggccaacccccaggccgagggccagctgcagtggctgaacaggagggccaacgccctgctggccaacggcgtggagctgagggacaaccagctggtggtgcccagcgagggcctgtacctgatctacagccaggtgctgttcaagggccagggctgccccagcacccacgtgctgctgacccacaccatcagcaggatcgccgtgagctaccagaccaaggtgaacctgctgagcgccatcaagagcccctgccagagggagacccccgagggcgccgaggccaagccctggtacgagcccatctacctgggcggcgtgttccagctggagaagggcgacaggctgagcgccgagatcaacaggcccgactacctgaacttcagggagagcggccaggtgtacttcggcatcatcgccctggtgaggagcagcagcaggacccccagcgacaagcccgtggcccacgtggtggccaacccccaggccgagggccagctgcagtggctgaacaggagggccaacgccctgctggccaacggcgtggagctgagggacaaccagctggtggtgcccagcgagggcctgtacctgatctacagccaggtgctgttcaagggccagggctgccccagcacccacgtgctgctgacccacaccatcagcaggatcgccgtgagctaccagaccaaggtgaacctgctgagcgccatcaagagcccctgccagagggagacccccgagggcgccgaggccaagccctggtacgagcccatctacctgggcggcgtgttccagctggagaagggcgacaggctgagcgccgagatcaacaggcccgactacctgaacttcagggagagcggccaggtgtacttcggcatcatcgccctg

SEQ ID NO: 37 - DNA, 2130 bp, synthetic construct
  source 1..2130: mol_type = other DNA; note = nucleic acid encoding SEQ ID NO:32 optimized for Musmusculus expression by www.jcat.de
gagcccaagagcagcgacaagacccacacctgccccccctgccccgcccccgaggccgccggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgccagcatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcggcggcggcagcgtgaggagcagcagcaggacccccagcgacaagcccgtggcccacgtggtggccaacccccaggccgagggccagctgcagtggctgaacaggagggccaacgccctgctggccaacggcgtggagctgagggacaaccagctggtggtgcccagcgagggcctgtacctgatctacagccaggtgctgttcaagggccagggctgccccagcacccacgtgctgctgacccacaccatcagcaggatcgccgtgagctaccagaccaaggtgaacctgctgagcgccatcaagagcccctgccagagggagacccccgagggcgccgaggccaagccctggtacgagcccatctacctgggcggcgtgttccagctggagaagggcgacaggctgagcgccgagatcaacaggcccgactacctgaacttcagggagagcggccaggtgtacttcggcatcatcgccctgggcggcggcggcagcagcagcaggacccccagcgacaagcccgtggcccacgtggtggccaacccccaggccgagggccagctgcagtggctgaacaggagggccaacgccctgctggccaacggcgtggagctgagggacaaccagctggtggtgcccagcgagggcctgtacctgatctacagccaggtgctgttcaagggccagggctgccccagcacccacgtgctgctgacccacaccatcagcaggatcgccgtgagctaccagaccaaggtgaacctgctgagcgccatcaagagcccctgccagagggagacccccgagggcgccgaggccaagccctggtacgagcccatctacctgggcggcgtgttccagctggagaagggcgacaggctgagcgccgagatcaacaggcccgactacctgaacttcagggagagcggccaggtgtacttcggcatcatcgccctgggcggcggcggcagcagcagcaggacccccagcgacaagcccgtggcccacgtggtggccaacccccaggccgagggccagctgcagtggctgaacaggagggccaacgccctgctggccaacggcgtggagctgagggacaaccagctggtggtgcccagcgagggcctgtacctgatctacagccaggtgctgttcaagggccagggctgccccagcacccacgtgctgctgacccacaccatcagcaggatcgccgtgagctaccagaccaaggtgaacctgctgagcgccatcaagagcccctgccagagggagacccccgagggcgccgaggccaagccctggtacgagcccatctacctgggcggcgtgttccagctggagaagggcgacaggctgagcgccgagatcaacaggcccgactacctgaacttcagggagagcggccaggtgtacttcggcatcatcgccctg

SEQ ID NO: 38 - DNA, 2103 bp, synthetic construct
  source 1..2103: mol_type = other DNA; note = nucleic acid encoding SEQ ID NO:34 optimized for Musmusculus expression by www.jcat.de
gtgaggagcagcagcaggacccccagcgacaagcccgtggcccacgtggtggccaacccccaggccgagggccagctgcagtggctgaacaggagggccaacgccctgctggccaacggcgtggagctgagggacaaccagctggtggtgcccagcgagggcctgtacctgatctacagccaggtgctgttcaagggccagggctgccccagcacccacgtgctgctgacccacaccatcagcaggatcgccgtgagctaccagaccaaggtgaacctgctgagcgccatcaagagcccctgccagagggagacccccgagggcgccgaggccaagccctggtacgagcccatctacctgggcggcgtgttccagctggagaagggcgacaggctgagcgccgagatcaacaggcccgactacctgaacttcagggagagcggccaggtgtacttcggcatcatcgccctggtgaggagcagcagcaggacccccagcgacaagcccgtggcccacgtggtggccaacccccaggccgagggccagctgcagtggctgaacaggagggccaacgccctgctggccaacggcgtggagctgagggacaaccagctggtggtgcccagcgagggcctgtacctgatctacagccaggtgctgttcaagggccagggctgccccagcacccacgtgctgctgacccacaccatcagcaggatcgccgtgagctaccagaccaaggtgaacctgctgagcgccatcaagagcccctgccagagggagacccccgagggcgccgaggccaagccctggtacgagcccatctacctgggcggcgtgttccagctggagaagggcgacaggctgagcgccgagatcaacaggcccgactacctgaacttcagggagagcggccaggtgtacttcggcatcatcgccctggtgaggagcagcagcaggacccccagcgacaagcccgtggcccacgtggtggccaacccccaggccgagggccagctgcagtggctgaacaggagggccaacgccctgctggccaacggcgtggagctgagggacaaccagctggtggtgcccagcgagggcctgtacctgatctacagccaggtgctgttcaagggccagggctgccccagcacccacgtgctgctgacccacaccatcagcaggatcgccgtgagctaccagaccaaggtgaacctgctgagcgccatcaagagcccctgccagagggagacccccgagggcgccgaggccaagccctggtacgagcccatctacctgggcggcgtgttccagctggagaagggcgacaggctgagcgccgagatcaacaggcccgactacctgaacttcagggagagcggccaggtgtacttcggcatcatcgccctggagcccaagagcagcgacaagacccacacctgccccccctgccccgcccccgaggccgccggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgccagcatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagcccc

SEQ ID NO: 39 - DNA, 2118 bp, synthetic construct
  source 1..2118: mol_type = other DNA; note = nucleic acid encoding SEQ ID NO:35 optimized for Musmusculus expression by www.jcat.de
gtgaggagcagcagcaggacccccagcgacaagcccgtggcccacgtggtggccaacccccaggccgagggccagctgcagtggctgaacaggagggccaacgccctgctggccaacggcgtggagctgagggacaaccagctggtggtgcccagcgagggcctgtacctgatctacagccaggtgctgttcaagggccagggctgccccagcacccacgtgctgctgacccacaccatcagcaggatcgccgtgagctaccagaccaaggtgaacctgctgagcgccatcaagagcccctgccagagggagacccccgagggcgccgaggccaagccctggtacgagcccatctacctgggcggcgtgttccagctggagaagggcgacaggctgagcgccgagatcaacaggcccgactacctgaacttcagggagagcggccaggtgtacttcggcatcatcgccctggtgaggagcagcagcaggacccccagcgacaagcccgtggcccacgtggtggccaacccccaggccgagggccagctgcagtggctgaacaggagggccaacgccctgctggccaacggcgtggagctgagggacaaccagctggtggtgcccagcgagggcctgtacctgatctacagccaggtgctgttcaagggccagggctgccccagcacccacgtgctgctgacccacaccatcagcaggatcgccgtgagctaccagaccaaggtgaacctgctgagcgccatcaagagcccctgccagagggagacccccgagggcgccgaggccaagccctggtacgagcccatctacctgggcggcgtgttccagctggagaagggcgacaggctgagcgccgagatcaacaggcccgactacctgaacttcagggagagcggccaggtgtacttcggcatcatcgccctggtgaggagcagcagcaggacccccagcgacaagcccgtggcccacgtggtggccaacccccaggccgagggccagctgcagtggctgaacaggagggccaacgccctgctggccaacggcgtggagctgagggacaaccagctggtggtgcccagcgagggcctgtacctgatctacagccaggtgctgttcaagggccagggctgccccagcacccacgtgctgctgacccacaccatcagcaggatcgccgtgagctaccagaccaaggtgaacctgctgagcgccatcaagagcccctgccagagggagacccccgagggcgccgaggccaagccctggtacgagcccatctacctgggcggcgtgttccagctggagaagggcgacaggctgagcgccgagatcaacaggcccgactacctgaacttcagggagagcggccaggtgtacttcggcatcatcgccctgggcggcggcggcagcgagcccaagagcagcgacaagacccacacctgccccccctgccccgcccccgaggccgccggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgccagcatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagcccc

SEQ ID NO: 40 - protein, 248 aa, Homo sapiens
  source 1..248: note = Human IgG1 sequence including C-terminal extension
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPELQLEESSAEAQDGELDG

SEQ ID NO: 41 - protein, 18 aa, Homo sapiens
  source 1..18: note = Linker from C-terminus of Human IgG
ELQLEESSAEAQDGELDG

SEQ ID NO: 42 - protein, 14 aa, Homo sapiens
  source 1..14: note = Linker variant derived from C-terminus of Human IgG
ELQLEESSAEAQGG

SEQ ID NO: 43 - protein, 22 aa, Homo sapiens
GGGSGGGTGSEFLASSRTPSDK

SEQ ID NO: 44 - protein, 25 aa, synthetic construct
  VARIANT 6..25: note = GGGGS repeats may be deleted
  source 1..25: note = linker
GGGGSGGGGSGGGGSGGGGSGGGGS

SEQ ID NO: 45 - protein, 50 aa, synthetic construct
  VARIANT 6..50: note = GGGGS repeats may be deleted
  source 1..50: note = linker
GGGGSGGGGSGGGGSGGGGSGGGGSGGGGSGGGGSGGGGSGGGGSGGGGS

SEQ ID NO: 46-99 - skipped (54 entries)

SEQ ID NO: 100 - protein, 592 aa, synthetic construct
  source 1..592: note = fusion protein
DFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNGGGSGGGTGSEFLASSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 101 - protein, 709 aa, synthetic construct
  source 1..709: note = fusion protein
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 102 - protein, 699 aa, synthetic construct
  source 1..699: note = fusion protein
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 103 - protein, 705 aa, synthetic construct
  source 1..705: note = fusion protein
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 104 - protein, 714 aa, synthetic construct
  source 1..714: note = fusion protein
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 105 - protein, 704 aa, synthetic construct
  source 1..704: note = fusion protein
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 106 - protein, 710 aa, synthetic construct
  source 1..710: note = fusion protein
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 107 - protein, 709 aa, synthetic construct
  source 1..709: note = fusion protein
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 108 - protein, 699 aa, synthetic construct
  source 1..699: note = fusion protein
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 109 - protein, 705 aa, synthetic construct
  source 1..705: note = fusion protein
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 110 - protein, 704 aa, synthetic construct
  source 1..704: note = fusion protein
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 111 - protein, 694 aa, synthetic construct
  source 1..694: note = fusion protein
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 112 - protein, 698 aa, synthetic construct
  source 1..698: note = fusion protein
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALVRSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALVRSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 113 - protein, 712 aa, synthetic construct
  source 1..712: note = fusion protein
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 114 - protein, 708 aa, synthetic construct
  source 1..708: note = fusion protein
ERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 115 - protein, 712 aa, synthetic construct
  source 1..712: note = fusion protein
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 708 aa, synthetic construct
  source 1..708: note = fusion protein
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 117 - protein, 694 aa, synthetic construct
  source 1..694: note = fusion protein
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 118 - protein, 693 aa, synthetic construct
  source 1..693: note = fusion protein
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 119 - protein, 698 aa, synthetic construct
  source 1..698: note = fusion protein
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 120 - protein, 704 aa, synthetic construct; identical to SEQ ID NO: 105
  source 1..704: note = fusion protein

SEQ ID NO: 121 - DNA, 2127 bp, synthetic construct
  source 1..2127: mol_type = other DNA; note = Codon optimized nucleic acid sequence encoding SEQ IDNO 101
gagtccaagtatgggccaccttgtccaccatgcccagcccccgaatttcttggtggcccttcagtctttctcttcccacccaaacccaaagatactcttatgatttctcgaacccccgaggtgacatgcgtggtcgtagacgtgagtcaggaagacccagaggttcagttcaactggtatgtcgacggcgtagaggtgcataacgccaagactaaaccccgagaagagcagtttaactccacttacagagtggtgagtgtcttgaccgtcctgcatcaggactggcttaacggcaaagagtataaatgtaaagttagcaataaaggactcccaagtagcattgaaaaaaccatcagtaaagcaaagggccaaccaagagagccccaggtgtatacccttccacccagtcaggaggaaatgaccaaaaaccaagtttcccttacttgccttgttaagggattctacccctcagacattgctgtagagtgggagtccaatggtcagcctgagaataattacaaaacaacacctcctgtgttggacagcgacggatctttctttctctatagtcgactcactgtggacaaatcaagatggcaggaggggaatgtgttctcatgctcagtaatgcatgaagccctgcacaatcactacacacaaaagagtctctctctgtcccttggaaagggtggaggtgggagcgtgcgctcttcaagccgcacaccatctgataagcctgtggcacatgtcgttgcaaatccacaagcagagggacaacttcagtggttgaacaggcgcgccaacgcattgctcgccaacggtgtcgagctgcgggacaaccagctggtcgtacctagtgagggtctgtacttgatctacagccaagtactgttcaaagggcagggctgtcccagcacccatgttctcttgactcataccatatcacgaatcgcagtaagttaccagactaaagtgaacctgctttccgctatcaaaagtccctgtcaaagagagactccagaaggggctgaggctaaaccttggtacgaaccaatttatctgggaggtgtgttccagcttgagaaaggagatcgcctttcagctgagatcaatcgaccagattatttgaattttcgagagagcggccaagtttattttggcataatcgcattgggtggtggtggtagctcctcacgcactccatctgacaagccagttgctcatgtcgtagctaatccccaggcagagggacaacttcaatggctgaacagaagggcaaacgccctgttggccaatggtgtggagttgagagacaatcagctggttgtcccttctgagggactttatcttatatatagccaagtgttgttcaaaggtcaagggtgcccctcaactcatgttctgttgacccataccataagtcgaatcgcagtgagttaccaaacaaaggtcaatctcttgtccgccataaagagcccctgccaacgggaaacacccgaaggagccgaggcaaaaccatggtacgaaccaatatacctcgggggagtgttccagctggagaagggagaccgactttcagctgaaatcaacaggcccgactatcttaacttcagggagtcagggcaggtctactttggaataatagcattgggcggaggcggatccagcagcagaactcctagcgacaagcccgttgctcatgtcgtagccaatccacaagccgaaggccagctgcagtggcttaatcgacgggccaatgccctgttggcaaacggagtcgagcttagggataatcagctcgttgttccaagtgaaggattgtatttgatctacagccaagttctgttcaagggtcagggttgcccctctacccatgttttgttgacacacacaatcagtcgcattgctgtatcctatcaaaccaaggtcaatttgctgtccgcaatcaagagcccatgccagagagagactccagaaggcgcagaagctaagccctggtacgagccaatttaccttggcggggttttccagcttgagaaaggagataggctgagcgcagaaatcaatcggcccgactacttgaatttccgcgaaagcggtcaagtgtattttggtatcatagcactt

SEQ ID NO: 122 - DNA, 2097 bp, synthetic construct
  source 1..2097: mol_type = other DNA; note = Codon optimized nucleic acid sequence encoding SEQ IDNO 102
gaatctaagtacggtcccccttgtccaccatgtccagcccccgagtttctcggagggcccagtgtctttcttttccctcctaaacccaaggatactctcatgattagccgaacacctgaagtaacatgtgttgttgtggacgttagtcaagaagaccccgaagttcaatttaactggtatgtggatggcgtagaggtacacaacgcaaagactaaaccacgagaagagcagttcaactccacttatcgagtagttagtgtgttgacagtactccatcaagactggctcaacggcaaagaatataagtgtaaagttagtaacaaaggactccccagtagcattgaaaagactatctccaaggcaaaagggcaaccaagggagccccaggtgtataccttgccaccctcacaagaggagatgacaaagaaccaggtcagtctcacctgtctggttaagggtttctatccttctgacattgccgttgaatgggagtctaacggccagcctgaaaataactacaagactacacctcccgtcctggatagcgatggtagttttttcctctattccaggctcactgtagacaagtcaaggtggcaggaaggcaatgttttcagctgctctgtcatgcatgaggcactccacaatcattatacacaaaaaagtctcagtttgtccttgggcaagggtggaggcgggagcgttcgcagctcctctcggactccaagcgacaaacctgttgctcatgtcgtcgccaatcctcaggcagaaggccaactgcaatggctgaacagacgcgctaatgcattgttggccaacggcgttgagttgagagacaaccaactcgttgtaccctccgagggactttatctgatatactctcaagtattgtttaagggtcaaggttgtccatcaacccacgtattgctgacccataccatttctagaattgccgtaagttatcagactaaagttaatttgttgagcgcaattaaaagtccttgtcaacgcgaaactcctgagggagcagaagcaaaaccctggtacgaacccatttatttgggaggggtatttcagctggaaaagggggatcggctgtcagccgaaattaatcgccctgattatctgaacttcagagaaagcggtcaagtctacttcggcatcatagccctttcatctcgcacaccaagtgataagcccgttgctcacgtcgtggcaaacccacaagccgaggggcaactccagtggttgaaccgcagggcaaatgctctcttggctaacggggtcgaattgagggataatcagctcgttgtcccttccgaaggactgtatctgatctacagccaagtactgttcaagggtcaaggttgcccaagtacacatgttttgctgacacatactataagccgcatcgccgtgtcttaccaaacaaaagtgaatctgctgtcagctataaagagcccatgtcagagggaaacacccgagggagctgaggcaaagccctggtacgaacccatatacttggggggcgtcttccaactggagaaaggtgacaggctcagtgcagagataaaccgccccgactacctgaattttcgagagagcggtcaagtatattttggtattattgcacttagtagtcggaccccatctgataaacccgtcgctcacgtcgtcgcaaacccacaagctgaggggcagttgcagtggcttaataggcgcgctaacgctctgcttgctaatggcgtggagttgagggataatcaattggtcgttcccagcgagggtctgtatttgatctacagccaggtactttttaagggccaaggctgccctagtactcatgtgcttctgactcatactatatcaaggatcgccgtcagctaccaaaccaaggttaatctccttagtgctatcaaaagcccatgtcaacgcgagactcccgagggcgccgaagccaaaccctggtacgagcccatatacctgggtggtgtgtttcagctggagaagggggaccgacttagtgcagagattaatagacctgattacctgaatttcagggagagcggtcaggtttattttgggatcatcgcactc

SEQ ID NO: 123 - DNA, 2115 bp, synthetic construct
  source 1..2115: mol_type = other DNA; note = Codon optimized nucleic acid sequence encoding SEQ IDNO 103
gagtcaaagtacggcccaccatgtcctccttgtcctgcccccgagtttctgggtggcccatccgtcttcctctttccacctaaaccaaaagataccctcatgatctctcggacacccgaagttacctgcgtcgtcgtcgacgtcagccaagaagatcctgaagttcagttcaattggtacgttgacggcgttgaggtacataacgccaaaacaaaaccccgggaggagcaattcaattctacttatcgggtggtttcagttttgaccgtgctgcatcaggactggctcaacgggaaagaatacaaatgtaaggtgtccaacaaaggactcccttccagtatagagaagactatatcaaaggccaagggccagccacgagagcctcaggtatacaccctgccccctagccaagaggagatgactaaaaaccaagtaagtctgacatgccttgtcaaggggttctatcctagtgatattgccgtagagtgggagtctaacggccagcccgagaacaattataagacaaccccacccgtgctggattcagatggatcttttttcttgtatagccggcttacagtagataaatctcgatggcaagaaggtaacgtgtttagttgctccgtaatgcacgaggcactccataatcactatactcaaaaatccctctccttgtctctgggcaaaggggggggcggctccgtccgatcatctagtcgcactccttcagacaagcctgtggcccacgtagttgctaatccacaggccgaggggcaactccaatggctcaaccgcagagccaacgcattgctggctaacggcgtagaattgcgagacaatcagcttgtggtaccttccgagggactgtacctcatctactctcaagttttgtttaaaggccaaggttgccccagtactcacgtacttctcactcacacaatcagccgcatcgctgtgtcttatcaaaccaaagtcaatttgctttccgccataaaaagcccttgtcagcgagaaacccctgaaggagctgaagctaaaccatggtacgagcccatctatctcggcggtgttttccagcttgagaagggggatcggctttccgccgagattaatcggcccgattacttgaatttcagggagagcgggcaggtgtattttggaataatcgctcttgtccggtcctcatctcgaacacctagtgataaacccgtagcccacgtagttgcaaatccccaggccgaaggtcaactgcagtggcttaaccgccgagcaaatgctcttctggcaaatggggtagagttgcgcgacaatcaattggtcgtaccaagtgaaggcctctaccttatctactctcaggtcctcttcaaaggtcaaggttgtccttctactcacgtactcctgacacatacaatatctcgcattgcagtatcataccaaacaaaggtgaatcttctctccgctataaaatcaccctgccaacgagagacacctgaaggtgcagaggccaaaccctggtacgaaccaatttaccttggaggagtttttcaattggaaaaaggagatagacttagcgccgaaataaataggcccgattacttgaattttagagagtccgggcaggtatatttcggcataatagcactggtcaggagttccagcaggactcccagcgataagcccgtcgcacacgtggttgctaatccacaagctgaaggacagctgcaatggcttaatagaagggccaatgctctgttggctaacggcgttgaacttcgggataaccagcttgtggtgccctccgaaggtttgtatttgatctattcacaagttttgttcaaaggccagggttgcccctctacccacgtacttctgacacacacaatcagccgcatcgctgtctcataccagaccaaagtcaacttgttgtctgcaataaaatcaccatgtcagcgggaaactcctgagggcgccgaggccaaaccctggtatgagccaatctaccttggtggcgtatttcagcttgaaaaaggagacaggctttccgcagagataaacaggccagattatctgaactttagggaatcaggtcaagtctactttggaatcatagctctc

SEQ ID NO: 124 - DNA, 2142 bp, synthetic construct
  source 1..2142: mol_type = other DNA; note = Codon optimized nucleic acid sequence encoding SEQ IDNO 104
gaatccaagtatggcccaccatgtcccccctgccccgcccctgaatttcttggcggacccagcgtatttctgttcccaccaaagcccaaggacacacttatgataagtcggacacctgaagtaacttgtgtcgtcgtcgacgtgagtcaggaagaccctgaagtccaatttaactggtacgtggatggcgtggaggtacacaatgccaagaccaagccacgcgaagagcagttcaattcaacatatcgggtcgtttccgtcctgaccgtactgcaccaagattggctcaatgggaaggagtacaaatgtaaagtatctaacaaaggcctcccatcctccatagaaaaaaccataagtaaagctaagggacagcctcgagaacctcaggtctacacactgcccccatctcaagaagaaatgaccaaaaaccaagtgagtcttacttgtctggtgaaaggtttctatccatccgacattgccgtagagtgggaatcaaacggccaacctgagaataactacaaaactactcctcccgtcctcgatagtgacggtagcttcttcctgtacagcaggctcacagtcgacaaatccaggtggcaagaaggcaatgttttcagctgttccgtcatgcatgaagccctgcacaaccattatacacagaaaagcttgagcctgtccttgggtaaaggtggaggggggagtgggggtggtgggtctgtgcgaagcagtagcagaacaccttccgacaaaccagttgcacatgttgttgctaatcctcaggccgaagggcagcttcagtggctcaacaggagggctaacgctttgttggctaacggtgtagagctccgcgataaccaacttgtagtgccttccgagggactctatcttatttactcccaagtgctgtttaaaggacaagggtgccctagcacccacgtattgctgactcacactatcagcaggattgccgtcagctaccagactaaagttaaccttctgtcagctataaaatcaccctgtcagcgggaaaccccagagggagcagaggcaaaaccctggtacgaaccaatatacttgggcggagtatttcaattggagaaaggtgatagactgagcgctgaaataaatcggcctgactatcttaacttccgcgaatcagggcaggtgtatttcggcatcattgccctcggtggcggagggagctcctcaaggactccaagcgataagccagtggctcacgtagtggccaatccacaagcagaaggtcaactgcaatggcttaaccgccgcgcaaacgcattgttggctaacggtgtggaattgagagataaccaattggtggttccttcagaaggcctgtacctgatctatagtcaagtactgttcaaaggacagggttgtcccagcactcatgttcttctgacccacactattagtagaatagccgtatcatatcaaaccaaagtcaaccttttgtctgccataaaatccccctgccaaagagaaacacccgaaggagccgaggccaaaccttggtacgagccaatatacctggggggcgttttccaattggaaaagggcgataggttgagcgctgagataaataggccagattatttgaatttcagggaaagcgggcaagtgtacttcgggatcatagccctgggcgggggtgggtcaagctctcgcactccctcagacaagcccgttgcacatgtggtggctaatccacaggctgagggacagctgcagtggctgaatagacgagcaaatgcactgcttgctaacggagttgagctccgcgataaccaactggtggtaccctctgagggactctatttgatttactcccaagttctcttcaagggccaaggctgcccctccactcatgtcctgcttacccacactatttctagaatagccgtatcttaccagaccaaggtcaacctcttgagtgcaataaagagtccctgtcaacgagaaactccagaaggcgccgaagctaagccatggtatgagccaatttacctcgggggagtgtttcagcttgagaaaggggacagactgagtgccgaaataaaccggcccgactatctcaacttccgcgagagtggtcaagtctacttcggtatcatagctttg

SEQ ID NO: 125 - DNA, 2112 bp, synthetic construct
  source 1..2112: mol_type = other DNA; note = Codon optimized nucleic acid sequence encoding SEQ IDNO 105
gagagtaaatacggcccaccttgtcctccctgccctgctccagagttccttggcgggccttccgtcttcctgtttccccccaagccaaaggacacactgatgatttcaagaaccccagaggtcacctgtgtcgttgtagatgttagtcaagaggatccagaggtgcaattcaattggtatgtcgatggggtggaggttcacaacgctaagaccaaacctcgggaagagcaattcaattctacttatcgggtggtaagtgttcttactgttttgcaccaggactggttgaacgggaaggaatataagtgcaaggttagtaacaaggggcttccttccagcatcgaaaagacaattagcaaagccaagggacaaccccgagagccacaagtgtatacccttcccccctcccaagaggaaatgaccaagaaccaagtctctctgacctgcctggtgaaagggttctatccaagcgacatagctgtcgaatgggaatccaacggccaacccgaaaataactataaaacaacacctcccgtcctggattccgatgggtcatttttcttgtattcaagattgaccgtggataaaagccgctggcaggaggggaacgttttttcatgtagtgtaatgcatgaagctcttcataaccattatacacagaaaagtttgagtttgtcactcggtaaaggtggaggagggtccggtggcggtggctcagtgagaagttcttctaggaccccttccgacaaacccgttgcccacgttgtcgcaaatccacaagctgaagggcagcttcagtggctcaatcggagagcaaatgctctccttgccaacggagtcgaactgcgcgacaaccaactcgtcgttccctccgagggcctgtatctgatctattcacaagtgttgttcaaaggtcaaggttgtccaagtacccatgtcttgctgacacacacaatatcaagaatagcagtcagctatcaaacaaaagtgaatttgctctctgccatcaaaagtccctgccaacgcgagactcctgaaggtgctgaagcaaaaccctggtatgaacctatatatttgggtggcgtctttcaacttgaaaagggtgacagactttctgccgagataaaccggccagactatctgaactttcgagagtccggtcaggtttatttcggtatcattgccttgagctctagaacacctagcgacaaacctgtcgcccatgtagttgcaaatccccaggctgagggtcaactccaatggcttaacaggcgcgccaacgctcttctcgccaacggtgtagagctgcgcgataatcaactggtggttccttccgagggactttatctgatatattcacaggttctgtttaaaggccagggttgtccctctacacatgtattgttgacacacactatatctcggatagctgtgagctaccaaacaaaagtaaatttgctgtctgctatcaagagtccatgtcagagggaaacccccgaaggagcagaggccaaaccatggtacgaaccaatatatcttgggggagtctttcaattggagaaaggggaccggttgagtgccgagattaaccgacctgattaccttaatttcagggagagcggtcaagtttacttcggcataatagccctttcttcacggacaccttcagacaaaccagtggctcatgtggttgcaaaccctcaagcagaaggtcaattgcaatggcttaatcgcagagctaatgcccttttggcaaacggtgtggagcttcgggataatcagttggtggttccaagtgaaggtctgtacttgatatattcccaagtgctgttcaaagggcagggctgcccctctactcatgttctgctcacccatacaatatctagaatcgctgtgagctaccagactaaggtcaatcttttgtcagcaataaaatcaccatgccaacgggagactccagaaggagcagaagccaaaccctggtatgaacctatatacctcgggggcgtctttcagcttgagaagggtgacaggctgagcgctgaaattaatcggcccgactaccttaactttagagaatccggtcaagtatatttcggtattattgccctc

SEQ ID NO: 126 - DNA, 2130 bp, synthetic construct
  source 1..2130: mol_type = other DNA; note = Codon optimized nucleic acid sequence encoding SEQ IDNO 106
gagagcaaatatggcccaccctgccccccatgtcctgccccagaattcctgggaggaccctcagtgtttctctttccacccaagccaaaagacacattgatgatttcaaggactcctgaggtgacatgtgttgtagtagacgtatcacaggaggatcctgaagtccagttcaactggtacgtcgacggcgttgaagtgcacaatgctaaaaccaagccccgagaggagcagtttaacagcacatatcgggtcgtttctgtgcttaccgtcttgcatcaggattggctgaacggaaaagaatataaatgcaaggtctcaaacaaggggcttccatcttcaatagaaaaaacaatttcaaaggcaaaaggacagcctagagagccccaagtctacactctgccacccagccaggaggagatgacaaagaaccaggtcagcctgacctgtctcgtcaaaggattctatccatccgacatcgccgtagaatgggagagtaatggacagcctgaaaacaactataagaccactcccccagtactggacagtgatgggtcattctttttgtatagtcgactgactgtagataaaagtcgatggcaggaaggtaatgtgttctcatgcagcgtcatgcacgaggccctgcacaaccattatacacagaagagtctgagtcttagcttgggtaagggaggcgggggatccggaggcggtggatctgtacggtcttctagcagaacaccaagtgataaaccagtggctcacgtggtagcaaacccccaagctgaggggcagcttcaatggcttaatagaagggctaacgctcttcttgccaacggggtcgagcttagggataaccagctggtggtcccctctgaaggcttgtatctgatatactcccaggtactgtttaaaggacaaggctgtcccagcactcatgtactgttgacacatactatatcacgcatagctgtctcttatcagacaaaagttaacttgcttagcgctatcaagagtccctgtcagagagaaacccccgaaggtgcagaggccaagccatggtacgaacctatttaccttggaggcgttttccaactggagaaaggggatcgcctctccgccgaaataaacaggcccgattatctgaacttccgagagagcggccaagtctactttgggataatcgctctcgtgcggagcagtagcagaaccccctctgataaaccagttgcccatgtggttgccaacccacaggccgaaggtcagctgcagtggctgaatcggagagccaacgctcttctcgccaatggtgtggaactcagggataaccaactggttgtcccatctgaaggtctttatcttatctattcacaagtgctctttaagggacagggctgtccaagtacacacgtcttgctcactcacacaatatccagaattgctgtaagctaccagacaaaagtaaacctccttagcgccattaaaagcccttgtcaaagggaaacacctgagggagccgaagccaaaccatggtacgaacccatatatctcggtggcgttttccagttggagaagggcgatcgactgtccgccgagattaatcgccctgattatctgaactttcgggagtccgggcaggtttactttggtataatcgcactggtacgctcaagcagtagaactccctcagacaaaccagtagcacatgttgtagctaatccacaagcagaaggacagctgcaatggctgaaccggagagctaacgccctgctggctaacggtgtcgagttgcgagataatcagcttgtcgtgcctagcgaggggctctaccttatttatagtcaagttctctttaaagggcaggggtgtccaagtacacacgtgttgctcacacatactatttctcgaatagccgtgtcctatcaaaccaaggtgaaccttctctccgctatcaaaagcccttgccaaagagaaacacccgaaggcgccgaggctaagccatggtacgaacctatctatctcgggggtgtttttcaactcgaaaaaggggacaggttgagtgctgagattaatagacccgattatttgaattttagggaatctgggcaggtttattttggaataattgctctc

SEQ ID NO: 127 - DNA, 2127 bp, synthetic construct
  source 1..2127: mol_type = other DNA; note = Codon optimized nucleic acid sequence encoding SEQ IDNO 107
gtacggagcagctctagaactccatctgacaagccagtcgctcatgtggtagcaaatccccaagctgagggccaacttcagtggttgaatcgcagggctaacgctctgctcgccaatggagtagaattgagggataatcagctcgtagtacctagcgaagggctttacctcatatattctcaggttctgtttaagggtcaaggctgtccaagtactcacgttctccttactcatacaatctctcgcatcgcagtttcttatcaaaccaaggttaatttgctgagcgccattaagtcaccatgccagcgcgaaacccccgaaggtgccgaagcaaaaccttggtatgagcccatttaccttggcggtgtgtttcagctggagaagggggacaggctttcagcagaaattaataggcccgactatcttaatttccgggagtccggccaggtttatttcggtatcattgccctgggcggtggcggctcatcctcacgcactccatctgataagcccgtcgcacatgtggtcgccaatcctcaggcagaggggcaattgcaatggcttaaccgcagggcaaacgctctgcttgctaatggggttgagcttcgggataaccagctcgtggtaccttcagagggtttgtacttgatctattctcaagtgcttttcaaaggacaaggttgcccaagcacccatgtgttgttgacccatactatttcccggatagcagtgtcatatcaaactaaggtcaatcttctgtcagctattaaaagtccctgtcagagagagactccagagggagctgaagccaaaccctggtacgagcccatatatcttggaggggtgttccagctcgagaaaggcgacagattgagcgccgagataaaccggcctgactatctcaattttcgagagtccggtcaggtttactttgggataatcgcactgggtggtggagggtctagctctcgcacaccatccgataagccagtagctcatgtggtggccaaccctcaagccgaggggcaacttcagtggctgaatagacgagctaatgcattgctggctaacggtgtcgaactgagagataatcagctcgtagtaccttcagaagggctttacctcatatactctcaggttttgttcaaaggacagggatgtccttcaactcacgtccttctcactcacactataagtagaatcgctgtatcctaccaaactaaagtgaaccttttgtctgctatcaaatccccttgccaacgcgaaactcccgaaggcgcagaagccaagccttggtatgagccaatctacctcggaggagtttttcagttggaaaagggtgacaggctgagtgctgaaatcaacaggcccgattatctgaacttcagggaaagcggacaagtgtattttggaataatcgcacttggtgggggagggtccgagtctaagtacgggccaccttgtcctccctgtccagcacctgagtttttgggcgggcccagtgtattcctgtttccacccaaacctaaggataccctgatgatatcacgaacccctgaggtcacctgtgttgtcgttgacgtaagtcaggaggacccagaggttcagttcaactggtatgtcgacggggtagaagttcataatgctaagactaagccaagggaggaacaatttaattccacttatcgagttgtgagcgtcctgacagttttgcatcaggattggcttaacggcaaagaatataagtgcaaggtttcaaataaaggtctgccttcttccatagaaaaaacaatctctaaagccaaaggccaaccaagagagcctcaggtgtacactcttcctccctctcaggaagagatgacaaaaaaccaggtgtccttgacctgtctcgttaaggggttctatccaagcgatattgctgttgagtgggaatcaaacgggcagcctgagaataattacaagaccacacccccagttttggatagcgatggtagtttcttcctttacagtaggttgaccgttgataagtcccggtggcaagaaggaaatgtgtttagttgctccgtgatgcacgaggcactgcataatcattacactcaaaagagtcttagtctgagcttggggaaa

SEQ ID NO: 128 - DNA, 2097 bp, synthetic construct
  source 1..2097: mol_type = other DNA; note = Codon optimized nucleic acid sequence encoding SEQ IDNO 108
gtgcggagtagcagcagaactccatccgataaaccagtggcacacgtggtcgctaatccccaagcagaagggcagctccaatggctgaacaggcgggccaatgcccttttggctaatggcgtcgagctcagagacaatcagctcgtcgtcccatctgagggtctctacttgatctatagtcaggtcttgttcaaaggccaaggctgtcctagtactcatgttctccttacacataccatttcaaggatagcagtctcatatcagactaaagtcaatctcctgagtgcaattaagtccccctgccagcgagagactccagaaggtgctgaggcaaagccatggtatgagcccatatatcttggcggagtctttcaactggagaagggtgaccggctctccgcagagattaaccggcctgactatctgaatttcagagagtctggccaggtttactttggcattatcgcactttccagtcggacccccagcgacaaacctgttgcccatgtcgtagcaaatccccaagccgaaggccagttgcagtggctgaacagacgagctaatgctttgttggcaaatggggtggagcttcgggacaatcaactcgtggtaccatctgaagggttgtacctgatatatagccaggtactctttaagggtcaaggttgtcctagtactcatgtgctcttgacccacacaatttcaagaatcgccgtcagttaccaaaccaaggttaatctgctttctgccataaagtctccctgccaacgcgaaaccccagaaggtgctgaagccaagccttggtacgagccaatctacctcggtggcgtttttcaacttgaaaagggggatcgcctgtctgccgagatcaacaggccagactacctgaacttccgagaaagtgggcaagtctattttgggatcatagccctgagctctcggacccccagcgacaagcctgttgcccacgtagttgctaaccctcaggctgaaggacaacttcagtggctgaacaggagagctaacgccctcctggctaatggagtcgaactgagagataatcaattggtcgtaccaagcgagggactgtacctcatatactctcaggtactgtttaagggccaaggatgtccaagtacccatgtacttctcacacatacaataagccggatagccgtcagctatcagactaaggtaaacctgctcagcgctattaagagcccatgccagcgagagaccccagaaggagcagaagctaaaccctggtacgagccaatatatcttggaggagtctttcaactggagaagggtgaccgattgagtgctgaaattaatcggccagattatttgaacttccgcgagagcgggcaagtgtatttcggaatcattgcacttggcgggggcgggagcgagtccaaatatggcccaccatgtcccccctgccctgccccagagttccttgggggcccttctgtatttctcttccccccaaaacccaaggatactcttatgatcagcaggactcctgaggtaacctgtgtggtcgtcgacgtatcacaagaggatccagaggtacagtttaattggtatgtagacggcgtggaagtccacaatgctaaaactaagcccagagaggagcagtttaatagtacataccgagtagtgagcgtattgactgtattgcatcaggactggttgaatgggaaagagtacaagtgcaaagtttccaacaaaggtctcccttcatctatcgagaaaaccatctcaaaggccaaaggccaacccagagagcctcaagtatacactctgccacccagccaagaagagatgactaagaatcaggttagtctcacttgtctcgtcaaagggttctatccctccgatattgctgtggaatgggagagcaacgggcaacccgagaacaactataagacaaccccaccagtacttgatagcgacgggtcttttttcctttattcacgccttacagttgataaatctcggtggcaggaagggaacgttttcagctgttctgttatgcatgaagccttgcataaccattacacacaaaagagtcttagtttgtctcttggaaag

SEQ ID NO: 129 - DNA, 2115 bp, synthetic construct
  source 1..2115: mol_type = other DNA; note = Codon optimized nucleic acid sequence encoding SEQ IDNO 109
gtgcgcagcagttccagaacacctagtgacaagcctgtggcacacgttgtggccaatcctcaagctgaaggtcagctccaatggcttaatagaagggctaacgcattgcttgctaatggggtggaacttcgagataaccaattggtggtgccctccgagggtctctaccttatctatagccaggtcctctttaaaggccaaggttgccccagtacacacgtcctgcttacacacacaatatccagaatagcagtctcataccagaccaaggtaaatctgcttagcgctattaagtcaccctgtcagcgggaaaccccagagggtgcagaagcaaaaccatggtatgagccaatttaccttggtggcgtttttcaactggaaaagggcgataggttgagcgccgagatcaatagacccgactatctcaattttcgggagtcaggccaggtttatttcgggatcattgctttggttcgctcctctagccgcaccccttccgataaaccagttgcacatgttgtggccaatccccaggctgaaggccagcttcagtggctcaacagacgggctaatgccctcctcgccaatggggtcgagctgagggacaaccaacttgtggtcccctcagaaggtctctaccttatctacagccaggttcttttcaaaggccagggctgtccttccactcacgtgctgttgacccataccatatcccgcattgccgttagctatcaaaccaaagtcaaccttttgtctgcaattaagagtccatgccagagagaaactcccgaaggtgcagaagcaaagccatggtatgaacctatatatctcggaggtgtgtttcaacttgagaaaggggacagactgagtgccgaaataaatcgccctgattatcttaatttccgagagtctgggcaagtatattttggaattattgccctcgtgcgaagctcttcaaggaccccaagtgataaacccgtagcacacgtagttgcaaatccacaagccgaaggacagttgcaatggctgaataggcgggctaatgctttgcttgctaatggggtcgagctgcgggataaccagcttgtcgtgccatctgaaggattgtacctgatatacagccaagttttgtttaagggacagggatgcccatcaacccacgtgctcctcactcacactatttctcgaattgccgtatcatatcagactaaagtcaacttgttgagcgcaataaagagcccttgtcaacgggaaacccccgagggtgcagaggccaaaccatggtatgaacctatttacctcgggggcgtctttcagttggaaaaaggtgatcggttgtccgctgagattaaccgaccagactatcttaactttcgggaatctggtcaagtctattttggcataattgcattggggggcgggggctctgaatccaaatacgggcctccttgccccccttgcccagcaccagaatttctcgggggcccatcagtttttcttttcccccctaagccaaaagataccctcatgatatcaagaactccagaggttacatgtgtcgtggtcgacgttagccaggaggatcccgaggttcagttcaattggtacgtggatggagttgaagtgcacaatgccaaaacaaaaccacgagaagagcaatttaatagcacctacagggtagtcagcgttcttacagttttgcaccaagattggcttaacggcaaagaatacaaatgtaaggttagtaataaaggactcccctcatcaatagaaaaaacaatttccaaagctaaaggccagcctagggaacctcaagtgtacacacttcctccaagtcaagaagagatgacaaagaaccaggtctcactcacttgtctcgtcaaaggtttctacccctctgacatcgccgtggaatgggagtccaatggccaacctgagaataattacaagaccacacctccagtactcgatagtgacgggtctttctttttgtattctaggttgacagtggataaatccagatggcaagaaggaaatgttttctcatgttctgtgatgcacgaggctcttcacaaccactacactcaaaagtctctgtctctttcccttggcaaa

SEQ ID NO: 130 - DNA, 2112 bp, synthetic construct
  source 1..2112: mol_type = other DNA; note = Codon optimized nucleic acid sequence encoding SEQ IDNO 110
gtccgatcatctagtaggacccctagcgacaagccagttgcacacgtggtagcaaacccacaagcagaaggacaactgcagtggcttaataggcgcgcaaatgcattgctcgccaatggagtggaactccgagacaaccaattggtagtgccttccgaaggactctaccttatttatagtcaggtcctgttcaaagggcaaggttgcccctcaacacacgtattgctgacacacaccatatcccgcatagcagttagctatcaaacaaaggttaatttgctgtccgcaataaagagcccctgccaacgggagacccccgagggcgcagaggcaaaaccctggtacgagcccatctacttgggtggcgtctttcaacttgaaaagggggataggctgagcgctgaaattaaccggcccgactatttgaatttccgggaatctggccaagtatactttggtattattgccttgggtggtggaggtagcagtagccgaacaccatcagacaaacctgtggcacacgttgtcgccaacccacaagctgaaggacaactccaatggttgaacaggcgagccaatgccctccttgcaaatggcgtagaattgcgagataatcagcttgttgttcctagcgagggtctttatcttatatacagtcaggtcctctttaaaggccaaggatgtcctagtacacacgtgctgctgactcatacaataagccgaattgccgtatcctatcagactaaggtcaaccttctgagcgctattaaatccccatgtcaaagggaaactccagaaggcgcagaagccaagccctggtatgagccaatctatctcggaggggttttccaattggagaagggcgaccggctttctgctgaaatcaatcgacctgattatctcaactttcgagagtcagggcaggtttatttcggtatcattgctctcggtggcggagggtccagctctaggaccccctcagacaaaccagtagcccacgttgtggccaatccccaggcagaaggtcagttgcagtggttgaatcggcgcgctaatgcactcctcgccaatggagttgaacttagggataatcaactcgtagtccccagcgaagggttgtatcttatttatagtcaggtcctttttaagggtcagggttgcccatccactcacgtgttgctcactcacaccatcagtcgcatcgccgtttcctatcagaccaaggttaatctcctgtccgctataaagtccccatgtcaaagagagacccccgaaggagcagaggcaaagccttggtacgagcctatatacttgggtggcgtatttcagttggaaaagggtgaccggttgtccgctgagataaatcgacctgactatctcaactttcgggagtctggtcaggtttactttgggattatagcactggagagcaaatacggacccccctgtcctccttgtcctgccccagagtttctcggtggaccatcagtctttctttttcctcctaagcccaaggatacattgatgatctcacggacccccgaagttacctgcgtggttgttgatgtaagtcaggaggatcccgaagtccaattcaattggtatgtcgacggcgtggaggtccacaatgcaaagacaaagccccgggaggaacagtttaacagcacataccgggtcgttagcgtgttgaccgtccttcatcaagattggttgaacggcaaagagtacaagtgcaaggttagcaacaaaggtttgccatcttccatcgagaaaacaatatctaaggccaaaggacagccccgcgaaccacaagtttatactcttcctccaagccaggaggaaatgactaagaatcaggtttccctcacatgccttgtaaagggtttttatccctcagatattgcagttgagtgggagagcaatggtcagcccgagaataactataaaacaaccccaccagtactcgactcagatggtagtttcttcctctactccaggttgacagtagacaaaagccgctggcaagagggcaacgtattctcttgctcagtgatgcatgaagcactgcataatcactacacacaaaaatctctgagcctttcacttggcaaa

SEQ ID NO: 131 - DNA, 2082 bp, synthetic construct
  source 1..2082: mol_type = other DNA; note = Codon optimized nucleic acid sequence encoding SEQ IDNO 111
gttaggtcttcatctagaacacccagcgacaagcccgtggcccacgtcgttgccaacccccaggcagagggtcagctgcagtggctcaataggcgagctaacgcccttctcgctaacggtgtggagttgcgcgataaccaactggtcgtaccatccgaaggactctatctgatttattctcaagtcctgtttaagggccagggctgtccttcaacccacgtcctccttacacataccatttctagaatagccgtatcatatcagactaaagtaaatcttttgtcagcaatcaaatctccatgccaacgggagaccccagagggagcagaagctaaaccctggtacgaacccatatatctgggcggtgtcttccagcttgagaagggggaccgactctcagccgagataaatcgacctgactatttgaacttcagagagtccgggcaagtctatttcggaattatagctctctcctctaggaccccatcagataaaccagttgcccatgtcgtggctaatccccaggctgaaggccaactgcaatggcttaaccgccgggccaatgctttgctcgccaacggtgtagagttgcgcgacaaccaactggtagtccctagcgaagggctgtacctgatctactcccaagttctttttaaaggccaaggttgtcctagtacccacgtacttctgacccatactatatctcggatagctgtgagttaccagacaaaggttaaccttctttccgccatcaaaagtccttgccaaagggaaacacctgaaggtgcagaagccaagccctggtatgagccaatttatctgggcggagtcttccaactcgagaagggggatagactgagcgctgagataaacagaccagactatctgaattttagggagtcaggccaggtatactttggaataatcgccctctcatcaaggactccctccgacaaaccagtagcacacgtagtggcaaatccccaggcagaaggacagctccagtggctgaatcggcgggcaaacgccctgctcgctaacggggtcgaacttagggacaaccagcttgttgtgccatccgaaggtttgtacctgatatattctcaagttctctttaaaggccaggggtgtccttctactcatgtgctgttgactcatacaatatcacggattgcagtttcctatcaaactaaagtaaacttgctttcagctatcaagagtccatgccaaagggagacacctgaaggggcagaggctaaaccctggtacgagcctatttacctcgggggcgtttttcagctggaaaaaggagatcggttgtcagctgaaatcaacagacccgactatctgaactttcgcgagtcaggtcaggtttattttggcattattgccctggaaagcaagtacggtcctccttgtccaccatgccctgctccagaattcttggggggaccatcagtgtttctgttcccccccaaaccaaaggacaccttgatgataagccgaaccccagaagtgacctgtgtcgtagttgatgtaagtcaagaagatccagaggtccaattcaactggtacgttgacggtgtcgaggtacataacgccaaaaccaagcctcgcgaagagcagtttaactccacatatagggtggtaagtgtgctcacagtgctgcatcaagactggcttaacgggaaggaatacaagtgtaaagtctccaataagggacttccctctagcatagaaaaaactatatctaaagcaaagggtcaaccacgcgaaccacaggtatatacactcccccctagccaggaggaaatgaccaaaaaccaagtatctttgacctgtctggtgaaaggcttttacccatctgatatcgcagttgaatgggagtcaaatggccaacccgaaaataactacaagacaactcctcccgtgctcgactctgacggatcattcttcctttactctcgcctcaccgtagataagagccgctggcaagagggtaacgtattcagttgtagcgtgatgcatgaggctcttcataaccattatacacaaaagtccctcagcctttctctgggaaag

SEQ ID NO: 132 - DNA, 2094 bp, synthetic construct
  source 1..2094: mol_type = other DNA; note = Codon optimized nucleic acid sequence encoding SEQ IDNO 112
gtccgctcatcatcaagaaccccaagcgacaaacctgtggcccacgttgttgccaatccacaagccgaggggcagctgcagtggcttaacaggagagcaaacgctcttcttgccaacggcgtagagcttcgagacaaccaacttgtcgtaccttctgaaggtctgtacctcatctatagtcaagtactttttaaaggacagggttgtccaagtacacatgtacttctgacccacacaatatccaggatagccgtgtcataccagacaaaggtcaatctgttgtctgcaattaagtcaccatgccaaagagaaaccccagaaggtgcagaagcaaagccatggtatgagccaatatatctgggcggcgtctttcagctcgagaagggagaccggctgtctgcagaaatcaacaggcctgactacctcaacttcagggagagtggccaggtgtattttggaataattgcattggttagaagttctcgcacaccatccgataaaccagtcgcccacgttgtagctaatccacaagccgagggacagctgcaatggctgaatcgacgggccaatgcattgctggctaatggggtagagcttcgcgataatcaacttgtggtcccatcagagggtctttacctcatatactcccaagtccttttcaaaggccaaggttgtccttctacacatgtgcttttgacccacactatttctagaatcgcagtgtcataccagactaaggtcaacctgctctcagctattaagtcaccctgccaaagggaaactcccgagggtgccgaggccaaaccttggtatgaacctatctaccttgggggagtgttccaactggagaagggcgatagattgagtgccgagataaatcggccagattatttgaacttcagagagagcggacaagtctacttcggtataatagcattggtgcgcagtagccgaactccctccgataagccagtcgcccatgttgtcgcaaaccctcaggcagagggacagcttcaatggctcaatcgccgcgccaatgccttgcttgccaacggtgttgaactgagggacaaccagttggtcgttcctagcgaaggtttgtatcttatctatagccaggtactgttcaaagggcaagggtgtcctagtacccatgtgctcctcacacataccatatcaagaattgcagttagttatcagaccaaggtaaatctcctgagtgcaataaaatccccctgccagcgggagactccagagggggctgaggccaaaccatggtacgagcccatctatctcggtggagtctttcagctggaaaagggagatcgcctttctgcagagattaataggccagattacctgaatttccgcgagagtgggcaagtttacttcggtatcatagcccttgaaagcaaatacggccctccatgccccccctgccctgcacccgagttcctgggcggtccctctgtgttcttgttccccccaaagcccaaggacaccctcatgatatccaggacaccagaagtaacttgcgttgtcgtcgatgtgtcccaggaagatccagaagttcaatttaactggtatgtcgatggtgtggaagtgcataatgcaaaaactaagcctcgagaagaacaattcaactctacatatcgcgtcgtcagtgtgttgactgtcctccaccaagactggctgaatggcaaagagtacaagtgcaaagtgtccaataagggccttccatcttcaattgagaaaaccattagtaaggcaaagggtcagccccgggaaccacaggtctatacattgccccctagccaagaggagatgaccaagaaccaagtctcactcacctgtctggtaaagggattttaccctagtgatatcgctgtcgaatgggaaagcaacggtcagcccgagaacaattacaaaaccactccaccagtgctcgactcagacggctcttttttcctttactcacggttgactgtagataaatcccgctggcaggagggcaatgttttcagctgtagtgttatgcacgaagcacttcacaatcattatacccagaagtcactgtctctttcccttgggaag

SEQ ID NO: 133 - DNA, 2136 bp, synthetic construct
  source 1..2136: mol_type = other DNA; note = Codon optimized nucleic acid sequence encoding SEQ IDNO 113
gagccaaagtccagcgacaagacacatacttgtccaccctgtccagctccagaggcagccggcggtccttccgtgttcttgtttcctcccaagccaaaggacacactgatgatctctagaactcccgaggttacatgcgttgtggttgacgtgtctcatgaggaccctgaagtgaagtttaattggtacgtcgacggtgtcgaggtacataatgcaaaaactaagccacgcgaggaacaatataatagcacataccgagtggtcagcgtcttgacagtgcttcaccaagactggctcaatggtaaggagtataaatgcaaagtatcaaacaaagccttgcccgcatccatcgaaaaaacaataagcaaggctaagggacaaccacgggagccacaagtgtatactctccccccttcaagagacgagctcacaaaaaaccaagtttcactgacttgcctggttaaaggtttttatccctccgatatagctgttgaatgggagagtaatggacaaccagaaaataactataaaactactcctcccgtgcttgacagtgacgggtcttttttcttgtattctaaactcaccgttgataaaagtagatggcagcagggcaatgttttctcctgctcagtgatgcatgaagctctgcacaatcactacacacaaaaatcactgtccctgtctcctggtaagggtggcggtggcagcgtcaggtcaagttccagaacacctagtgataaaccagtagcccatgtagttgctaacccccaggctgagggacaacttcagtggcttaaccgccgcgctaatgctcttcttgctaacggagtcgaactgagagataaccaacttgtcgtgcctagtgaggggttgtatctcatttactctcaggtgctgttcaagggccagggctgtccatcaactcacgtactgcttacacatactattagcaggatagcagtgagctaccaaaccaaagttaacttgttgtctgccattaaaagcccttgtcagagggaaacccctgagggggcagaagctaagccatggtacgaacctatttaccttggtggggtgtttcagttggagaaaggggatcggcttagtgctgaaataaatagacccgattatttgaacttccgggagagtggtcaggtttacttcggaatcatcgccctgggaggggggggttctagctcaaggacaccaagcgataaaccagtggcacatgtggtcgctaatccccaagcagaggggcaacttcagtggttgaaccgccgggctaatgcactgctcgcaaacggtgtagagttgagggacaatcaactcgttgtaccaagtgagggcttgtatctcatatacagccaggtgctttttaaaggccaggggtgtcccagtacacacgtgttgctcacccacacaatatcaagaatagcagtctcataccaaactaaggttaatctcctctcagcaattaaatccccttgtcagcgggagacccccgaaggcgctgaggctaagccctggtacgaacccatctatcttggtggggtttttcaactggagaaaggcgatcgattgtcagccgagattaatcgcccagattacctgaactttcgcgaatccggacaggtatacttcggcattatcgcattgggtggcggtggcagcagcagtaggactcctagcgataaacccgttgctcatgttgttgcaaacccacaggcagaagggcagctccaatggctcaatcggcgcgcaaacgcattgctggccaacggagtagagctgcgggacaaccaacttgttgttcccagcgaaggtctttacctcatttattcccaagtccttttcaagggccaaggctgtccaagtacacacgtacttcttactcacacaataagtcgcatagcagtctcttaccaaacaaaagtcaatctcctgtctgcaattaaatccccatgtcaaagagaaaccccagaaggggcagaggccaagccttggtatgagcctatctatttgggcggggttttccaacttgagaagggagaccggctttcagctgaaatcaacaggcccgattatctcaacttcagggagagtggacaagtctacttcggaattatagccctg

SEQ ID NO: 134 - DNA, 2124 bp, synthetic construct
  source 1..2124: mol_type = other DNA; note = Codon optimized nucleic acid sequence encoding SEQ IDNO 114
gaaaggaagtcaagcgtggaatgccctccctgtccagcaccacccgtcgctggacccagcgtgttcctgttcccacccaaacccaaggatactctcatgatcagccggacaccagaggtaacttgtgtagtagtagatgttagccatgaggatcctgaggtgcagtttaattggtacgttgacggggtggaggtacataacgcaaaaaccaaaccacgagaggagcagttcaacagcacctttcgcgtagtgtcagtcctgaccgtagtccaccaggactggttgaacggtaaggaatacaagtgtaaggtttccaacaagggtctgcctgcctctatcgagaaaacaataagcaagacaaaaggccaacctcgggaacctcaggtatatacacttcccccaagtcgagaggagatgactaagaaccaggtaagccttacttgcctggtaaaaggtttttatcccagcgacatcgccgtcgaatgggaatccaatggacagcctgagaataactataagacaaccccccctatgctggattcagacggtagcttctttctttattccaaacttaccgtggataaatcaaggtggcagcaagggaatgttttctcttgtagtgtcatgcacgaagcccttcacaaccattacactcagaaatccctcagcttgtcacctggaaaagggggcggcggaagtgtccgatcctcctctcggaccccatctgacaagccagttgcccatgtggtggctaatccacaggctgaggggcaactccagtggctgaataggagagctaatgctctccttgctaatggagttgaacttagagacaatcagcttgtcgtcccctctgaagggctctatttgatatacagccaggttctttttaagggtcagggctgtccctccactcatgtgcttctcacacacacaatcagccgcatcgcagtgagttatcaaaccaaagttaacctgctttccgcaatcaaaagcccttgtcagagagaaaccccagaaggagcagaagccaaaccctggtatgagcccatctatctcggaggagtattccaactggaaaagggtgataggttgagcgctgagataaatagacccgactatctgaacttcagggagagtggtcaagtatactttggcattattgccctcggcggcggcggcagttccagtcggacaccctcagataagccagttgctcacgttgtggccaacccccaagccgaaggccagttgcagtggttgaataggcgggctaatgctctgctggcaaacggtgtagaacttcgagataatcaactcgttgtgccctcagagggactctatctcatttacagccaggtgcttttcaaagggcaggggtgtccctctacacatgtccttctgacacatacaatctcacgaatagctgtctcctaccaaacaaaagttaatttgctcagtgctataaaatccccttgccagcgggagacacctgagggggctgaggccaaaccttggtacgagcctatctatctcggcggggtattccaacttgaaaaaggggacagacttagtgccgaaataaaccgcccagactaccttaacttccgcgagtccgggcaggtttactttgggataatcgcactggggggaggtggatcttcatctagaaccccaagcgacaaaccagttgctcatgtggtcgccaatcctcaagctgaaggacagcttcaatggcttaatcgccgggcaaacgcccttttggcaaatggcgttgagctgcgggataatcaactggtagttccaagtgagggcttgtacttgatctatagtcaagtactgttcaagggccaaggctgcccatctacacacgttcttttgacccacactatttcaaggattgccgtcagctatcaaactaaagtgaacctcctgtctgctatcaagtcaccctgtcaacgagaaacccctgagggtgctgaagccaagccctggtatgagcccatatatctcggcggagtctttcaactggagaagggtgacaggctgtctgccgaaatcaatcggcctgactatctgaactttcgggagagcggccaggtctacttcggcattattgctctc

SEQ ID NO: 135 - DNA, 2136 bp, synthetic construct
  source 1..2136: mol_type = other DNA; note = Codon optimized nucleic acid sequence encoding SEQ IDNO 115
gtcaggagtagctctaggaccccatccgataagcccgtcgcacatgtggtggccaacccccaggcagaaggccaactccagtggcttaatagacgagccaatgcccttttggctaatggcgtcgagctcagggacaatcaacttgtggtgcctagtgagggactctatttgatttatagccaagtacttttcaagggacagggttgtccatctacacacgtgcttcttacccacactatttctcggatcgcagtttcttatcaaaccaaagtcaaccttttgtccgctatcaagagtccatgtcagagagagacacccgagggcgctgaagctaagccctggtatgagccaatctatcttgggggagttttccagctcgaaaaaggggaccggctgtctgccgaaattaaccgccctgactacctcaactttagggagagtggtcaggtgtatttcggaataatcgccttgggcggtggcgggtcatctagcagaaccccatccgacaagccagtcgcccatgtagtggccaatccacaggcagagggacaattgcagtggttgaatcggcgagccaatgcattgctcgcaaacggggtggagctccgcgataaccagcttgtagtgccatccgaaggattgtatttgatttattctcaagtgctgttcaaaggacaagggtgcccatctacccatgtcttgctgacacacacaatttcccggatcgctgtatcctaccaaaccaaggtgaatcttttgtcagcaatcaaaagcccatgtcaacgcgaaacaccagagggagcagaggccaagccttggtacgagcctatttacctgggcggtgtctttcaacttgagaagggagatcgcttgagcgcagaaattaataggcctgactaccttaactttagggaaagtggacaggtatattttggaataattgcactcggtggtgggggatcatcaagccgcacaccttccgataaacccgttgcccacgtagtggcaaatccccaggccgaaggccaattgcaatggctgaaccgaagagccaacgctcttctcgcaaatggtgtagaactccgggataaccagttggtggtgcccagcgagggcctttatctcatatactctcaagtccttttcaaagggcagggatgtcctagtacccatgtacttctcactcacacaatctccaggatcgccgtttcatatcaaacaaaagttaatttgctcagcgctataaagagtccatgccaacgcgaaacacctgagggggccgaagcaaaaccttggtacgagcctatttatcttggtggagtattccaacttgaaaaaggtgacaggttgtcagctgagattaatagaccagattatctgaattttcgcgaatctgggcaggtttacttcgggataatcgctctgggaggaggagggagtgaaccaaagtccagcgataaaactcatacctgtccaccttgtccagcccccgaagctgcaggaggccctagcgtgttcttgtttcctcccaaacccaaagacacattgatgattagtcgcactcctgaagtgacatgtgttgtcgtagacgtatctcatgaagaccccgaagtcaagtttaactggtatgtcgatggagtggaggtgcacaatgcaaagactaagcctagggaagaacaatataacagtacctacagagttgtgtcagtgcttaccgtcttgcatcaggattggctcaatggaaaagagtataagtgtaaggtaagtaacaaggcattgcccgctagcatagagaaaacaataagcaaggcaaaggggcagcccagggaaccccaagtctatacccttccaccaagtcgggatgaactgactaaaaatcaggtgtccttgacttgccttgtaaagggattctacccctcagatatcgcagtggagtgggagagcaacggacagccagaaaacaattacaaaaccaccccccctgtcctggattcagacggttctttctttttgtactccaaacttacagtagacaagtccaggtggcaacaaggcaatgtctttagctgttctgtcatgcacgaagcccttcacaaccactatactcaaaagtcactttctctttcccctggaaaa

SEQ ID NO: 136 - DNA, 2124 bp, synthetic construct
  source 1..2124: mol_type = other DNA; note = Codon optimized nucleic acid sequence encoding SEQ IDNO 116
gtaagatcatctagtcggactccatcagacaaaccagtagcccatgttgttgcaaacccacaagccgagggtcaacttcagtggctcaataggcgcgccaatgcactgctcgctaatggagtcgaattgcgcgataaccaattggtggtacctagtgagggactttatttgatctatagtcaggtgctctttaaaggtcagggttgcccctccacccacgttctcctgacacataccattagcaggatagctgtaagttaccagactaaagtcaacctccttagcgctatcaaaagtccatgtcaaagagaaactccagaaggagcagaagccaaaccttggtacgagcctatctacctcggaggagtatttcagcttgaaaagggggatcgactgagcgccgaaatcaacagacccgattaccttaacttccgagaatccggccaagtatacttcgggattattgcccttgggggaggtggctcttcaagcagaaccccatcagacaagccagtggctcacgtcgttgccaatccccaagctgaagggcaacttcaatggcttaatcgaagggctaatgcacttttggccaacggtgtagaactccgagacaaccaattggtcgtgccatcagaaggcctttacctcatatactcccaggttcttttcaagggtcagggatgtcctagtacacacgtattgttgacccatacaatttcaaggatagcagtaagctaccagactaaagttaatctgcttagtgctataaagtctccttgtcagcgagagacacccgaaggcgctgaggcaaaaccctggtacgagcccatctacctcgggggtgtttttcaactggagaagggagaccgactgtccgccgaaattaaccggcccgactacctcaattttcgcgaatccgggcaagtttattttggtatcattgcattgggtggtggaggctccagtagccggactccctccgataaaccagtggcacatgtagtcgccaaccctcaagcagaagggcaattgcagtggctgaatagacgcgccaatgccctcctggctaatggcgtagagcttagagataatcaattggtggtgcctagtgaaggtctgtacctcatttactctcaggttctctttaagggccaaggatgtccctcaactcacgtactgctgactcatactatatcacggatagccgtctcttaccagacaaaagtgaatttgctgtcagccatcaagagtccatgccagcgagaaacccctgagggggctgaagctaaaccatggtatgaaccaatctaccttggtggcgttttccagctcgagaagggcgatagacttagcgccgaaattaatcgaccagactatctcaattttagagagtcaggacaagtgtactttggtattatagccttgggtgggggcggttctgaacggaaaagttctgttgaatgccctccatgtcctgccccccctgtggccggtccctcagtctttctcttcccacccaagcccaaagatacattgatgattagtaggactcccgaggtgacttgcgtagttgtcgatgtttcccatgaagatccagaagtgcaatttaactggtatgtagacggcgtcgaggtccataatgctaaaactaagccccgcgaggagcagtttaattcaacctttagagttgtgagcgttctgaccgttgtacaccaggattggcttaatggtaaagagtacaagtgcaaggtgtccaacaagggacttccagcatccattgaaaagaccatttccaagactaaagggcaaccacgggaaccacaagtctacaccctcccacccagccgcgaagagatgactaaaaatcaggtatcacttacttgcctggttaagggtttctacccatctgacattgctgtcgagtgggaatctaatgggcaacctgaaaacaattacaagacaacaccacctatgctggattccgatgggagtttcttcctgtacagtaaactcactgttgacaagtcccgatggcagcagggaaatgtcttttcatgctccgttatgcatgaggccctccacaaccattatacccaaaagtctctgtccctgtcaccaggaaag

SEQ ID NO: 137 - DNA, 2082 bp, synthetic construct
  source 1..2082: mol_type = other DNA; note = Codon optimized nucleic acid sequence encoding SEQ IDNO 117
gaatcaaagtacggtccaccttgtcctccctgtcccgcccccgagtttctggggggtccctctgtctttctgtttccaccaaagcccaaggacactctgatgattagcagaacaccagaagtaacctgtgtcgtcgtggatgtctcacaggaggatcccgaggtacagttcaactggtacgtggatggtgtagaggtgcataatgcaaagactaaaccaagggaagaacaattcaattctacttaccgggtcgtatctgtcttgaccgtgcttcaccaagattggctgaacggcaaggagtataaatgtaaagtttctaataaggggctcccatcaagtatcgagaaaaccatttcaaaagcaaaagggcaacctcgagagcctcaagtttacacactccctccatcacaagaagaaatgacaaagaatcaagtcagcctcacctgccttgtaaagggcttctatccctccgacattgcagtggaatgggagtcaaacggacaacctgagaataattataagaccacacctccagtgctggactcagatgggtcatttttcctgtactcccgcttgaccgtggacaagtctcgatggcaggaaggtaatgtgttcagctgtagtgtgatgcacgaagcactgcacaaccattatacccagaaatccctgtcattgtccctcggtaaggtgagatccagtagccgcacaccaagtgataaacctgtagcccacgtagtggcaaatccacaagctgaagggcagctccagtggctgaatcgccgcgcaaacgcactgctggcaaatggggtagagcttagggacaatcagctcgtagtgcccagtgaaggcctctatctcatttattcacaagtacttttcaaaggccagggatgccctagtacccatgtccttttgacacacaccatctcccgaatagccgtaagctaccaaactaaggttaatctccttagcgcaatcaaatctccttgccaaagggaaacccccgaaggcgccgaagccaagccctggtatgaacctatataccttggcggggtttttcagctggaaaagggagacaggttgagtgccgagattaatcgaccagactaccttaattttagagagtccggccaggtctatttcgggataatcgctctgtcttctagaactcccagtgataaacccgttgcccacgtggtggccaacccacaggccgaagggcaactgcagtggctgaacagacgagcaaatgcattgttggccaacggtgttgaactgcgcgacaaccaacttgtggtgcctagtgagggtctctacttgatttattcccaagtcctctttaaagggcaagggtgtccctctactcatgtcctgctcactcacaccatctccagaattgcagtatcttatcagacaaaagtaaacttgctgtcagccattaaatcaccatgtcagagggagacacctgaaggtgcagaagctaagccttggtatgaacctatttatctcggcggggtgttccaattggagaaaggggaccgactgagcgctgaaatcaatagacccgattatttgaactttagagagagtggccaggtatacttcggtataatagccctgtccagtcgaactccttctgataagcctgtcgcacatgttgtggcaaatcctcaagctgagggacagctccaatggttgaatagacgcgccaacgcactcctcgctaacggggttgagctccgagacaatcagcttgtcgtcccaagcgaggggctgtaccttatttactcccaggtattgtttaagggacagggttgcccctccacacatgtgctcctgacccacactatcagccgaatagccgttagctatcaaacaaaggtcaatctcctgagtgcaataaagtctccttgtcagcgagaaacccccgaaggcgccgaggccaagccctggtacgagccaatttacctcggtggagtctttcagttggagaagggggatagattgagcgcagaaattaaccgacctgactatttgaacttcagagaaagcggacaagtctattttggtatcatcgccctg

SEQ ID NO: 138 - DNA, 2079 bp, synthetic construct
  source 1..2079: mol_type = other DNA; note = Codon optimized nucleic acid sequence encoding SEQ IDNO 118
gaatcaaagtacggccctccatgtccaccctgtcctgcccctgagtttctcggaggacccagtgtattcctcttcccaccaaaacccaaggataccctcatgatcagcaggactcccgaagttacatgcgttgtcgtagacgtatcacaggaagatcctgaggtccaatttaattggtacgtcgacggagtcgaagttcataacgccaaaacaaaaccacgagaagagcaatttaacagtacatatcgcgtggtctcagtgctgaccgtgctccaccaggactggctcaatgggaaagaatacaaatgtaaggtttccaataagggactccctagctcaatagaaaagaccatttcaaaagctaaaggccaaccccgggagccccaagtctacacccttcccccctctcaggaagaaatgaccaaaaatcaggtgtccctgacctgtcttgtgaaagggttttatccctcagacattgccgtagagtgggaatcaaatggacaacccgagaacaactataaaactactccacctgttctggactccgatggttcctttttcctgtacagccgccttaccgttgacaaatcacgatggcaggaagggaatgtcttcagttgttcagtaatgcatgaagctctccataaccactatactcagaagtccctgtccctctctctgggcaagggcggcggtggttccgtccgcagttcttctcggactccctccgacaagccagtcgcacatgtagtcgccaacccacaagcagagggacagcttcagtggctcaatcgaagagcaaacgccctccttgcaaacggcgtcgaacttcgcgacaaccaactggttgttccatcagaaggcttgtatctgatctactctcaggtgctgtttaagggacagggatgtcctagcacacatgtgctccttactcatacaatttcaaggatcgcagtaagctaccaaactaaagtgaacctccttagcgccataaagtccccatgccaaagggagacacccgagggagcagaagcaaagccatggtatgaacctatctatctcggtggagttttccagttggagaaaggtgatagactctctgctgagatcaatcgccccgactatctgaatttccgcgaatctgggcaggtctactttgggataatagcactgggtggcggtggatctcccgtagctcacgtggtcgctaacccacaggctgaggggcaattgcaatggttgaaccggcgggctaatgctttgttggcaaacggcgtagaattgagagacaaccaattggtcgttccttcagaaggattgtatctcatctacagccaagtcttgtttaaaggccaaggctgtccatctacacacgtgcttcttactcacacaatctcacgaatcgcagtatcttatcagaccaaagtgaacttgctctctgcaataaaaagcccttgtcaacgcgaaactccagaaggggctgaagcaaagccatggtacgaacctatttatctcgggggggtgttccaactcgagaaaggggaccgactgtccgctgaaatcaaccgccctgactatcttaatttccgggagtctgggcaggtatatttcggtataattgcacttggaggcggggggtcacctgtggcacatgtagtcgccaacccccaagctgaaggacaacttcaatggctcaataggcgcgcaaatgctctgctcgcaaatggagtagaactccgggataatcaactggttgtgccttctgaaggactgtatctgatctatagccaagttttgttcaagggccaggggtgcccatctacacacgtacttcttacccacacaatatcccgcatcgccgtcagttatcagacaaaagtgaaccttttgtccgccatcaagagcccatgtcagcgcgaaactcccgagggtgctgaggctaaaccatggtatgagcccatctatttgggaggcgtatttcaactggaaaaaggggatcgactgagcgcagagatcaataggcccgattatcttaatttcagggagtctggtcaagtgtattttgggataattgctctg

SEQ ID NO: 139 - DNA, 2094 bp, synthetic construct
  source 1..2094: mol_type = other DNA; note = Codon optimized nucleic acid sequence encoding SEQ IDNO 119
gaatctaagtatgggccaccatgcccaccatgcccagccccagaattcctgggcggaccttccgttttcttgttcccaccaaagccaaaagatactctgatgatttccaggacccctgaagttacctgtgtggtagtggatgtcagccaggaggatccagaagttcaatttaattggtatgtagacggagtcgaagtccataacgctaaaactaaacctcgagaagagcagtttaattcaacctacagggttgtttccgtactgacagttttgcatcaggactggctgaatggcaaggaatacaaatgcaaggtcagcaacaaaggactcccaagttcaatagaaaagaccatttcaaaagctaaagggcaaccacgagaacctcaggtctacactctccctccctctcaggaagagatgactaaaaatcaggtttcacttacatgcctcgtgaagggcttttaccccagcgacattgctgttgagtgggagagtaacggacaacctgagaacaactacaagactacacctcctgtgctggactcagatggttccttctttttgtatagcaggcttaccgttgataagtcccgctggcaagaaggcaacgttttcagttgttcagtaatgcacgaagctctccacaatcattatacacagaagagtcttagcctgtccctgggtaagggaggcggggggtccgggggcgggggctcagttcgctcatcaagccgaacaccctcagacaagccagttgcccacgtcgtagccaacccccaagctgaaggacagttgcaatggctgaataggcgagctaatgcattgttggcaaatggagtagaactgcgcgataatcaattggttgtgccctcagaagggctgtaccttatttactcccaggtgctcttcaaagggcagggttgcccttcaacccacgtacttcttacccacacaataagcaggattgccgtctcctaccaaactaaagtaaacctgttgagcgctatcaagagtccttgccaacgggagacccctgaaggtgcagaggcaaaaccatggtacgaacccatttatctcggaggggtgttccagttggagaagggggaccgcctgtctgccgaaatcaataggccagactacctcaactttcgcgagtccgggcaggtgtattttgggatcatagctttgggcggtgggggatctcctgttgctcatgtcgttgcaaaccctcaggctgaaggccaattgcaatggctcaacaggagagctaacgcattgctggccaacggggttgagctccgcgataaccagctggtagttccctcagagggcttgtaccttatctattcacaggttctcttcaaaggacaaggatgtcctagcacacacgtcttgcttacacataccattagccggatagcagtttcttatcagactaaagttaatctcctctctgccataaagtcaccctgtcagcgggaaacacctgagggtgctgaagcaaaaccttggtatgaaccaatatacctcggtggagtttttcaactggagaagggcgacagactgagcgctgaaataaacagacctgactaccttaatttccgagaatcaggtcaagtatacttcgggattatagccttggggggtggaggctccccagtggctcatgtagtcgctaatccccaagctgaaggccaactccaatggcttaacaggagggccaacgcactcctcgcaaatggagtcgagcttagggataatcaattggtggttccctctgagggcttgtatcttatttattcacaggtcctgtttaaaggccaaggctgtccttctacacatgtcctgttgactcataccataagtagaatagccgtgagttaccagacaaaggttaacctgctttccgcaatcaaatctccatgccaacgcgagaccccagaaggggcagaagcaaagccttggtacgagcccatatatctcggtggggtctttcagctcgagaaaggcgaccggcttagcgctgaaatcaaccgcccagactatttgaactttcgggaaagtggacaagtctacttcggtatcatagcactc

SEQ ID NO: 140 - DNA, 2112 bp, synthetic construct
  source 1..2112: mol_type = other DNA; note = Codon optimized nucleic acid sequence encoding SEQ IDNO 120
gaatctaagtatggacctccttgtccaccatgtccagctcccgagttcctgggaggcccatccgtgtttttgttcccccctaagccaaaagacacacttatgatatcaagaaccccagaagttacttgtgtagtcgtggacgtatcccaggaagaccccgaggttcaatttaactggtatgtagacggcgtggaagtccataatgctaagacaaagccccgggaggaacaattcaactccacataccgagtagtatccgtattgaccgtgctccatcaggattggttgaatggaaaggaatacaagtgcaaagtttctaataagggcctgccttctagcatcgagaagaccatcagcaaggctaagggacagcctcgcgaaccccaagtttatacccttcctcctagccaagaggagatgactaaaaatcaggtgtcactcacctgcctcgtcaaaggattctacccatcagatatagcagtggaatgggagtccaacgggcaacctgagaataactacaaaacaactccacctgtcctggactccgacggctccttctttctttattccagacttaccgtggacaaaagcagatggcaagaggggaatgtgtttagctgcagtgttatgcatgaagctttgcataatcattacacccaaaaatcactttcactctctcttggtaaggggggtgggggatctggtgggggaggctccgtgcgatcaagctctaggacaccctctgataaacctgttgcccacgtcgtcgcaaatccccaggccgaaggacagttgcagtggctgaatcgaagagctaacgcactgttggcaaacggggtggagctcagggataaccagttggtggtgccttcagaagggctttatctcatttactcacaagtactctttaaagggcaagggtgcccatctactcacgtgttgctgactcacactatttctcgaatcgcagttagctatcaaaccaaggtaaacttgctcagtgccataaaaagtccttgtcaaagggagacacccgaaggagcagaagcaaagccctggtacgagcccatttacctcggtggtgtcttccagctggagaaaggagaccggctctctgcagagataaacagacctgactatctcaactttagagaatcaggccaggtttatttcgggatcatcgcactctccagccggaccccctcagacaagcccgttgcacacgtcgttgctaacccacaagctgaagggcagttgcagtggttgaatcgaagagcaaacgctctcttggccaacggtgtagaactccgcgacaaccaactggttgtaccttcagaagggctctatctgatttactctcaggtgcttttcaagggccaagggtgccctagtacacatgttctgcttacccacacaatttctagaattgcagttagctaccagactaaagtcaacctgttgagtgctatcaagtccccttgtcagagagaaaccccagagggagctgaggctaaaccttggtatgagcccatatacctcggtggtgtattccaattggagaaaggtgatcgattgtcagctgaaatcaacagaccagactatctgaatttcagagagtcaggacaagtttacttcggcataatcgcattgagtagtcggacaccctccgataaacctgtggcacatgttgtagctaaccctcaagcagaggggcagctccaatggctgaaccggcgcgctaatgccctgttggctaacggcgttgagttgcgagataaccagctggttgtgccctctgaaggtctgtacttgatctactcccaagtcctgtttaagggtcaaggctgtcccagcacacacgtgttgctcacccacactatcagccggattgccgtaagctatcaaactaaagtcaatcttctgtccgccatcaaaagtccatgtcagcgcgaaacccctgagggtgccgaagccaagccttggtacgagccaatctacctgggtggcgtctttcagctcgaaaagggggaccggctctctgcagagataaatcgccctgattatcttaactttcgcgagtccgggcaggtatactttgggattatagctctt

(19) United States
(12) Patent Application Publication
(10) Pub. No.: US 2026/0116938 A1
(43) Pub. Date: Apr. 30, 2026

(54) SINGLE-CHAIN TNF RECEPTOR 2 AGONIST FUSION PROTEINS

(71) Applicant: Relinia, Inc., Euless, TX (US)

(72) Inventors: David C. Foster, Euless, TX (US); Lutz B. Giebel, San Mateo, CA (US); Leonard G. Presta, San Francisco, CA (US)

(21) Appl. No.: 19/242,321

(22) Filed: Jun. 18, 2025

Related U.S. Application Data
(63) Continuation of application No. 18/052,180, filed on Nov. 2, 2022, now Pat. No. 12,365,712.
Application No. 18/052,180 is a division of application No. 16/618,233, now Pat. No. 11,530,247.
(60) Provisional application No. 62/515,643, filed on Jun. 6, 2017.

(51) Int. Cl. C07K 14/525; A61K 38/00; A61K 39/00; A61P 29/00; C07K 16/46; C07K 19/00; C12N 5/07; C12N 15/62

(52) U.S. Cl. CPC C07K 14/525; A61P 29/00; A61K 38/00; A61K 39/00; C07K 16/46; C07K 19/00; C07K 2319/02; C07K 2319/30; C07K 2319/50; C07K 2319/70; C12N 5/06; C12N 15/62

(57) ABSTRACT

This invention provides for a fusion protein between a single chain TNFR2 Selective Agonist protein (seTNFR2 Selective Agonist) and a dimerization domain, such as an IgGFc protein. The single chain TNFR2 Selective Agonist moiety provides a theapeutic activity by selectively activating the TNFR2 form of the TNF-α receptor, thus selectively stimulating Tregs and/or increasing myelin deposition.

### CROSS REFERENCE TO RELATED APPLICATIONS

This application is a continuation of U.S. application Ser. No. 18/052,180, filed Nov. 2, 2022, now U.S. Pat. No. 12,365,712, which is a divisional of U.S. application Ser. No. 16/618,233, filed Nov. 29, 2019, now U.S. Pat. No. 11,530,247, which is a National Stage Entry of International Patent Application No. PCT/US2018/036139, filed Jun. 5, 2018, which claims the benefit of, and priority to, U.S. Provisional Application No. 62/515,643, filed Jun. 6, 2017, the entire contents of which are incorporated herein by reference.

### STATEMENT REGARDING FEDERALLY FUNDED RESEARCH

None

### REFERENCE TO SEQUENCE LISTING

A listing of the sequences follows the specification and is expressly included in or incorporated herein by reference. This application contains a Sequence Listing which has been submitted electronically in XML format and is hereby incorporated by reference in its entirety. Said XML file, created Oct. 8, 2025, is named TBI-100USD1C1_SL.xml and is 179,033 bytes in size.

### BACKGROUND

### I. Field of the Invention

The present invention relates generally to the fields of TNF Receptor 2 agonist molecules and uses thereof.

### II. Description of Related Art

Tumor necrosis factor-a (TNF-a) is a cytokine that is responsible for diverse biological effects such as inflammation and immune modulation. It is a target of a variety of therapeutic agents including antibodies such as Humira and Remicade.

### SUMMARY

In one embodiment the present disclosure provides a fusion protein comprising a first TNF homology domain (THD) comprising D143N/A145R mutations, wherein the THD has at least 95% identity to SEQ ID NO: 3; a second THD comprising D143N/A145R mutations, wherein the THD has at least 95% identity to SEQ ID NO: 3; a third THD comprising D143N/A145R mutations, wherein the THD has at least 95% identity to SEQ ID NO: 3; an immunoglobulin Fc domain; and a first linker peptide covalently linking the first and second THDs and a second linker covalently linking the second and third THDs.

In some embodiments the linkers in the fusion protein are composed of from 1-31 or 2-15 or 3-10 amino acids and in some embodiments include at least some stalk region from TNF-a.

In some embodiments the Fc in the fusion protein is covalently linked to the N-terminus of the N-terminal THD or the C-terminus of the C-terminal THD.

In some embodiments the Fc is covalently linked to the THD by a linker, although in some embodiments the Fc and THD are directly connected.

In some embodiments the TNFR2 agonist-Fc fusion protein selectively activates TNFR2 over TNFR1, and in some embodiments upon administration to a subject, this fusion protein selectively activates a TNFR2 in the subject over TNFR1 in the subject. In some embodiments the TNFR2 agonist-Fc fusion protein preferentially activates T regulatory cells in the subject relative to conventional T cells in the subject. In some embodiments the TNFR2 agonist-Fc fusion protein increases myelination in a subject compared to control administration.

In some embodiments the present disclosure provides a nucleic acid encoding a fusion protein as described above.

In some embodiments the present disclosure provides a method of increasing myelin deposition in a patient in need thereof comprising administering a fusion protein as described herein to said patient.

In some embodiments the present disclosure provides method of treating demyelinating disease in a patient in need thereof comprising administering a fusion protein as described herein to said patient. In some embodiments the demyelinating disease is optic neuritis or multiple sclerosis.

In some embodiments the present disclosure provides a method of treating pain in a patient in need thereof comprising administering a fusion protein as described herein to said patient.

It is contemplated that any embodiment of a method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device or method being employed to determine the value.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-1G: Configurations of scTNFR2 agonist fusion proteins. FIG. 1A shows domains of scTNFR2 agonist fusion proteins. FIG. 1B shows a scTNFR2 agonist fusion protein comprising N-terminal Fc, linker 1, stalk sequence, THD, linker 2, stalk sequence, THD, linker 2, stalk sequence THD. FIG. 1C shows a scTNFR2 agonist fusion protein comprising N-terminal Fc, linker 1, stalk sequence variation, THD, linker 2, stalk sequence variation, THD, linker 2, stalk sequence, THD. FIG. 1D shows a scTNFR2 agonist fusion protein comprising N-terminal Fc, linker 1, THD, linker 2, THD, linker 2, THD. FIG. 1E shows a scTNFR2 agonist fusion protein comprising N-terminal Fc, linker 1, stalk sequence, THD, linker 2, THD, linker 2, THD. FIG. 1F shows a scTNFR2 agonist fusion protein comprising N-terminal Fc, stalk sequence, THD, linker 2, THD, linker 2, THD. FIG. 1G shows a scTNFR2 agonist fusion protein comprising N-terminal THD, linker 2, THD, linker 2, THD, linker 1, Fc.

FIG. 2 Depicts sequence of wild type TNF-a. (SEQ ID NO:1) Bold indicates ADAM17 cleavage site between A and V. Italics indicate stalk region (amino acids 57-87). Underline indicates THD (amino acids 88-233). Arrows indicate amino acids to be mutated to form TNFR2 agonist.

FIG. 3 Depicts sequence of mature, soluble TNF-a. (SEQ ID NO:2).

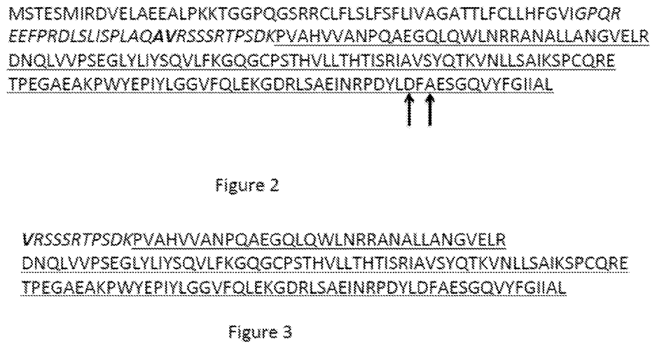

FIG. 4 Depicts the TNF homology domain (THD) containing D143N/A145R mutations. (SEQ ID NO:3).

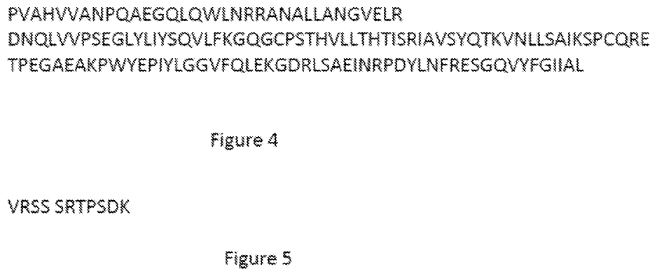

FIG. 5 Depicts the sequence from the ADAMI7 cleavage site in the stalk region to the C-terminus of the stalk region. (SEQ ID NO:4).

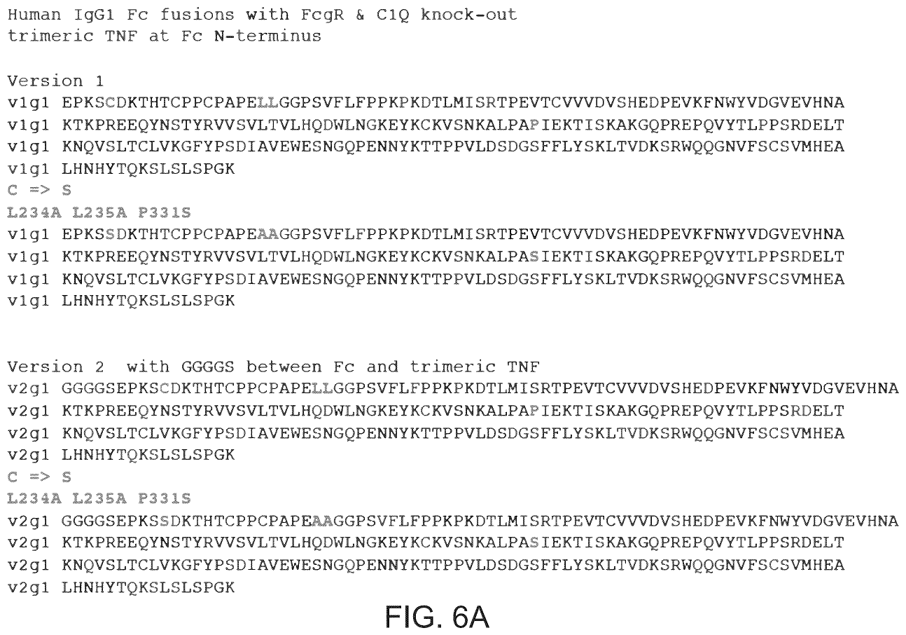

FIG. 6A Version 1-Depicts Human IgG1 Fc sequence (SEQ ID NO:5) with FcyR and Clq knockout (SEQ ID NO:6). The C-terminus of the scTNFR2 agonist can be fused directly to Fc N-terminus. Version 2-Depicts Human IgG1 Fc sequence like Version 1 with the exception that linker GGGGS (SEQ ID NO: 25) is placed between the N-terminus of the Fc and C-terminus of the scTNFR2 agonist. (SEQ ID NO:7 and SEQ ID NO:8).

FIG. 6B Version 3-Depicts Human IgG1 Fc sequence with FcyR and C1Q knockout. The scTNFR2 agonist is at the Fc C-terminus contains a spacer of (GGGGS)ₙ, wherein n=1=5 (SEQ ID NO: 44). (SEQ ID NO:9 and SEQ ID NO: 10).

FIG. 7A Version 1-Depicts Human IgG4 Fc sequence. (SEQ ID NQ: 11) and a variant containing Ser to Pro mutation (SEQ ID NO: 12) The C-terminus of the scTNFR2 agonist can be fused directly to Fc-N-terminus. Version 2-Depicts Human IgG4 Fc sequence like Version 1 with the exception that linker GGGGS (SEQ ID NO: 25) is placed between the N-terminus of the Fc and C-terminus of the scTNFR2 agonist. (SEQ ID NO: 13 and SEQ ID NO: 14).

FIG. 7B Version 3-Depicts Human IgG4 Fc sequence. The scTNFR2 agonist is at the Fc C-terminus which contains a spacer of (GGGGS)ₙ, wherein n=1=5 (SEQ ID NO: 44). (SEQ ID NO: 15 and SEQ ID NO: 16).

FIG. 8A Version 1-Depicts Human IgG2 Fc sequence (SEQ ID NO:17) with Clq knockout (SEQ ID NO: 18). The C-terminus of the scTNFR2 agonist can be fused directly to Fc-N-terminus. Version 2-Depicts Human IgG2 Fc sequence like Version 1 with the exception that linker GGGGS (SEQ ID NO: 25) is placed between the N-terminus of the Fc and C-terminus of the scTNFR2 agonist. (SEQ ID NO: 19 and SEQ ID NO:20).

FIG. 8B Version 3-Depicts Human IgG2 Fc sequence with C1Q knockout. The scTNFR2 agonist is at the Fc C-terminus contains a spacer of (GGGGS)ₙ, wherein n=1=5 (SEQ ID NO: 44). (SEQ ID NO:21 and SEQ ID NO:22).

FIG. 9 Depicts human IgG sequence including a C-terminal extension (SEQ ID NO:40).

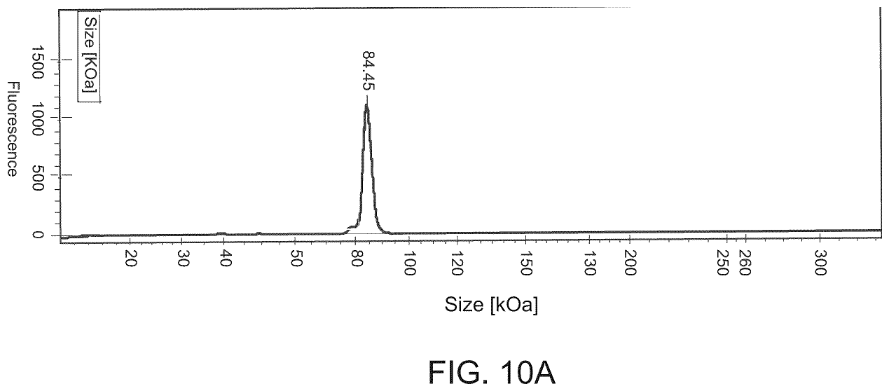

FIGS. 10A-10B: FIG. 10A shows an electrophoregram of SEQ ID NO: 101 under non-reducing conditions. FIG. 10B shows an electrophoregram of SEQ ID NO: 101 under non-reducing conditions.

FIGS. 11A-11B: FIG. 11A shows binding of TNF Variants to immobilized TNFR1. FIG. 11B shows binding of TNF Variants to immobilized TNFR2.

FIGS. 12A-12B: FIG. 12A shows Kym-1 Cell Viability assay in the presence of TNF variants. FIG. 12B shows Kym-1 Cell Viability assay in the presence of TNF variants.

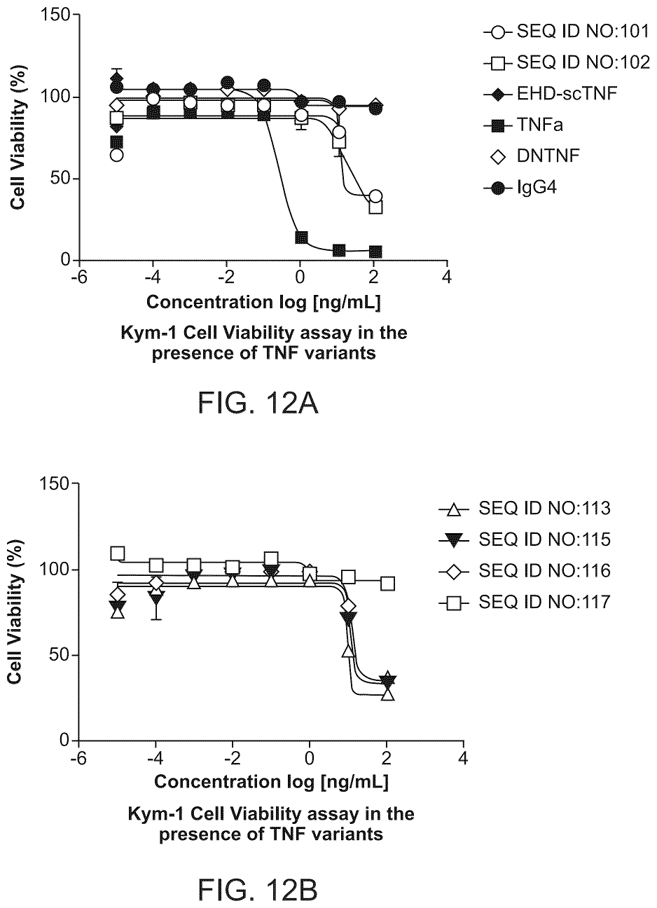

### DESCRIPTION

TNF-a is found in both soluble forms and transmembrane forms as a homotrimer. The transmembrane precursor is cleaved, resulting in soluble form. The soluble and transmembrane form signal through two distinct receptors, TNFR1 and TNFR2, resulting in distinct biological effects. Soluble TNF-a (sTNF-a) signaling through TNFR1 is thought to mediate inflammation while transmembrane TNF-a (tmTNF-a) signaling through TNFR2 is thought to modulate immune response, stimulation of regulatory T-cells (Tregs) and myelin regulation.

While current products and methods of inhibiting TNF-a are effective and account for a significant therapeutic market, the current therapies are not without deleterious side effects. These range from immunosuppression to demyelination of neurons. For instance, therapeutics that are effective immunomodulators in the periphery are contraindicated for treatment of neuro inflammatory disorders. Currently marketed TNF-a inhibitors are labeled with a BLACK BOX WARNING specifically warning against treatment of neurological diseases because they cause demyelination resulting in worsening of the condition. These current TNF-a inhibitors block signaling by both soluble and tmTNF-a, resulting in the beneficial anti-inflammatory effects but also leading to deleterious side effects. Accordingly, there is a significant need for the development of molecules that stimulate signaling through the TNFR2 but not TNFR1.

Accordingly, the present disclosure provides novel TNFR2 agonist molecules. These find use as improved compositions and methods for treating disorders such as, but not limited to pain, nerve injury and/or demyelinating diseases such as, but not limited to multiple sclerosis and optic neuritis.

### Definitions

“At least a percent (eg. 97%) sequence identify to Sequence ID No. X” as used herein refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci USA 67:22264-2268, 1990) modified as in Karlin and Altschul, Proc. Natl. Acad. Sci USA 90:5873-5877, 1993 is incorporated into the NBLAST and XBLAST programs of Altschul et al. (Altschul, et al. J. Mol. Biol. 215:403-410, 1990). To obtain gapped alignments for comparison purposes, Gapped BLAST is utilized as described in Altschul et al. (Altschul, et al. Nucleic Acids Res. 25:3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL world-wide web address of: “ncbi.nlm.nih.gov” for these programs. In a specific embodiment, percent identity is calculated using BLAST2 with default parameters as provided by the NCBI.

“N-terminus” refers to the end of a peptide or polypeptide that bears an amino group in contrast to the carboxyl end bearing a carboxyl acid group.

“C-terminus” refers to the end of a peptide or polypeptide that bears a carboxylic acid group in contrast to the amino terminus bearing an amino group.

“C-terminal IgG Fc protein moiety” refers to a portion of a fusion protein that derives from two identical protein fragments, each having a hinge region, a second constant domain, and a third constant domains of the IgG molecule's two heavy chains, and consisting of the carboxy-terminai heavy chains disulphide bonded to each other through the hinge region.

It is functionally defined as that part of the IgG molecule that interacts with the complement protein Clq and the IgG-Fc receptors (FcyR), mediating Complement-dependent cytotoxicity (CDC) and antibody-dependent cellular cytotoxicity (ADCC) effector functions. The sequence can be modified to decrease effector functions, to increase circulating half-life, and to eliminate glycosylation sites.

### Single-Chain TNF-a Variants

The single chain TNF-a variant fusion proteins described herein are generally composed of contiguous amino acids having the following domain structure:

DD-L1-THD-L2-THD-L3-THD or THD-L2-THD-L3-THD-L1-DD, where DD is a dimerization domain as described herein. LI, L2 and L3 are linkers that may be the same or different and THD is a TNF-a homology domain as defined herein. In preferred embodiments the fusion protein is encoded by contiguous nucleotides and expressed as a single contiguous polypeptide.

“N-terminal human TNF-a variant protein moiety” or “N-terminal scTNFR2 Agonist (scTNFR2)” refers to an N-terminal domain of a fusion protein that is derived from a wild type TNF-a protein structurally and functionally defined herein and that is composed of three THDs.

“C-terminal human TNF-a variant protein moiety” or “C-terminal scTNFR2 Agonist (scTNFR2)” refers to a C-terminal domain of a fusion protein that is derived from a wild type TNF-a protein structurally and functionally defines above.

### Tregs

“Tregs” or “Treg cells” refer to Regulatory T cells. Regulatory T cells are a class of T cells that suppress the activity of other immune cells, and arc defined using flow cytometry by the cell marker phenotype CD4+CD25+FOXP3+. Because FOXP3 is an intracellular protein and requires cell fixation and permeablization for staining, the cell surface phenotype CD4+CD25+CD127− can be used for defining live Tregs. Tregs also include various Treg subclasses, such as tTregs (thymus-derived) and pTregs (peripherally-derived, differentiated from naive T cells in the periphery).

### Peptide Linkers

“Peptide linker” is defined as an amino acid sequence located between the two proteins comprising a fusion protein, such that the linker peptide sequence is not derived from either partner protein. Peptide linkers are incorporated into fusion proteins as spacers in order to promote proper protein folding and stability of the component protein moieties, to improve protein expression, or to enable better bioactivity of the two fusion partners (Chen, et al., 2013, Adv Drug Deliv Rev. 65 (10).1357-69). Peptide linkers can be divided into the categories of unstructured flexible peptides or rigid structured peptides.

### Fc Fusion Proteins

An “Fc fusion protein” is a protein made by recombinant DNA technology in which the translational reading frame of the Fc domain of a mammalian IgG protein is fused to that of another protein (“Fc fusion partner”) to produce a novel single recombinant polypeptide. Fc fusion proteins are typically produced as disulfide-linked dimers, joined together by disulfide bonds located in the hinge region.

### Functional Activation

“Bioactivity” refers to the measurement of biological activity in a quantitative cell-based in vitro assay.

“Functional activation of Treg cells” is defined a TNF-a-mediated response in Tregs. Assay readouts for functional activation of Treg cells includes stimulation of pSTAT5, Treg cell proliferation, and stimulation of the levels of Treg effector proteins.

### Design and Construction

There are multiple options for the design and construction of an Fc fusion protein, and the choices among these design options are presented below to permit the generation of a molecule with the desired biological activity and pharmaceutical characteristics. Key design options are: (1) the nature of the TNF-ot Selective Agonist, (2) the choice of the dimerization domain protein moiety, i.e. Fc, (3) the configuration of fusion partners in the fusion protein, and (4) the amino acid sequence at the junction between the dimerization domain and the fusion partner protein as well as between the three THDs.

### General Methods

In general, preparation of the fusion proteins of the invention can be accomplished by procedures disclosed herein and by recognized recombinant DNA techniques involving, e.g., polymerase chain amplification reactions (PCR), preparation of plasmid DNA, cleavage of DNA with restriction enzymes, preparation of oligonucleotides, ligation of DNA, isolation of mRNA, introduction of the DNA into a suitable cell, transformation or transfection of a host, culturing of the host. Additionally, the fusion molecules can be isolated and purified using chaotropic agents and well known electrophoretic, centrifugation and chromatographic methods. See generally, Sambrook et al., Molecular Cloning: A Laboratory Manual (2nd ed. (1989); and Ausubel et al., Current Protocols in Molecular Biology, John Wiley & Sons, New York (1989) for disclosure relating to these methods.

The genes encoding the fusion proteins of this invention involve restriction enzyme digestion and ligation as the basic steps employed to yield DNA encoding the desired fusions. The ends of the DNA fragment may require modification prior to ligation, and this may be accomplished by filling in overhangs, deleting terminal portions of the fragment(s) with nucleases (e.g., ExoIII), site directed mutagenesis, or by adding new base pairs by PCR. Polylinkers and adaptors may be employed to facilitate joining of selected fragments. The expression construct is typically assembled in stages employing rounds of restriction, ligation, and transformation of E. coli. Numerous cloning vectors suitable for construction of the expression construct are known in the art (X.ZAP and pBLUESCRIPT SK-1, Stratagene, LaJolla, Calif., pET, Novagen Inc., Madison, Wis.—cited in Ausubel et al., 1999) and the particular choice is not critical to the invention. The selection of cloning vector will be influenced by the gene transfer system selected for introduction of the expression construct into the host cell. At the end of each stage, the resulting construct may be analyzed by restriction, DNA sequence, hybridization and PCR analyses.

Site-directed mutagenesis is typically used to introduce specific mutations into the genes encoding the fusion proteins of this invention by methods known in the art. See, for example, U.S. Patent Application Publication 2004/0171154; Storici et al., 2001, Nature Biotechnology 19:773-776; Kren et al., 1998, Nat. Med. 4:285-290; and Calissano and Macino, 1996, Fungal Genet. Newslett. 43:15-16. Any site-directed mutagenesis procedure can be used in the present invention. There are many commercial kits available that can be used to prepare the variants of this invention.

Various promoters (transcriptional initiation regulatory region) may be used according to the invention. The selection of the appropriate promoter is dependent upon the proposed expression host. Promoters from heterologous sources may be used as long as they are functional in the chosen host.

Various signal sequences may be used to facilitate expression of the proteins described herein. Signal sequence are selected or designed for efficient secretion and processing in the expression host may also be used. A signal sequence, which is homologous to the TCR coding sequence or the mouse IL-2 coding sequence may be used for mammalian cells. Other suitable signal sequence/host cell pairs include the B. subtilis sacB signal sequence for secretion in B. subtilis, and the Saccharomyces cerevisiae a-mating factor or P. pastoris acid phosphatase phol signal sequences for P, pastoris secretion. The signal sequence may be joined directly through the sequence encoding the signal peptidase cleavage site to the protein coding sequence, or through a short nucleotide bridge.

Elements for enhancing transcription and translation have been identified for eukaryotic protein expression systems. For example, positioning the cauliflower mosaic virus (CaMV) promoter 1000 bp on either side of a heterologous promoter may elevate transcriptional levels by 10- to 400-fold in plant cells. The expression construct should also include the appropriate translational initiation sequences. Modification of the expression construct to include a Kozak consensus sequence for proper translational initiation may increase the level of translation by 10 fold.

The expression cassette(s) are joined to appropriate vectors compatible with the host that is being employed. The vector must be able to accommodate the DNA sequence coding for the fusion proteins to be expressed. Suitable host cells include eukaryotic and prokaryotic cells, preferably those cells that can be easily transformed and exhibit rapid growth in culture medium. Specifically preferred hosts cells include prokaryotes such as E. coli. Bacillus subtillus, etc. and eukaryotes such as animal cells and yeast strains, e.g., S. cerevisiae. Mammalian cells are generally preferred, particularly HEK, J558, NSO, SP2-O or CHO. Other suitable hosts include, e.g., insect cells such as Sf9. Conventional culturing conditions are employed. See Sambrook, supra. Stable transformed or transfected cell lines can then be selected. In vitro transcription-translation systems can also be employed as an expression system.

Nucleic acid encoding a desired fusion protein can be introduced into a host cell by standard techniques for transfecting cells. The term “transfecting” or “transfection” is intended to encompass all conventional techniques for introducing nucleic acid into host cells, including calcium phosphate co-precipitation, DEAE-dextran-mediated transfection, lipofection, electroporation, micro injection, viral transduction and/or integration. Suitable methods for transfecting host cells can be found in Sambrook et al. supra, and other laboratory textbooks.

Alternatively, one can use synthetic gene construction for all or part of the construction of the proteins described herein. This entails in vitro synthesis of a designed polynucleotide molecule to encode a polypeptide molecule of interest. Gene synthesis can be performed utilizing a number of techniques, such as the multiplex microchip-based technology described by Tian, et. al., (Tian, et. al., Nature 432:1050-1054) and similar technologies wherein oligonucleotides are synthesized and assembled upon photo-programmable microfluidic chips.

The fusion proteins of this invention are isolated from harvested host cells or from the culture medium. Standard protein purification techniques are used to isolate the proteins of interest from the medium or from the harvested cells. In particular, the purification techniques can be used to express and purify a desired fusion protein on a large-scale (i.e. in at least milligram quantities) from a variety of approaches including roller bottles, spinner flasks, tissue culture plates, bioreactor, or a fermentor.·

### The TNFR2 Selective Agonist Moiety and Fusion Proteins

In one embodiment the molecules described herein are single-chain, trimeric TNF-a molecules. By “single-chain” is meant that a single polypeptide comprises 3 THDs as described herein.

The single chain TNF-a variant fusion proteins described herein are generally composed of contiguous amino acids having the following domain structure: DD-L1-THD-L2-THD-L3-THD or THD-L2-THD-L3-THD-L1-DD, where DD is a dimerization domain as described herein. LI, L2 and L3 are linkers that may be the same or different and THD is a TNF-a homology domain as defined herein. In preferred embodiments the fusion protein is encoded by contiguous nucleotides and expressed as a single contiguous polypeptide.

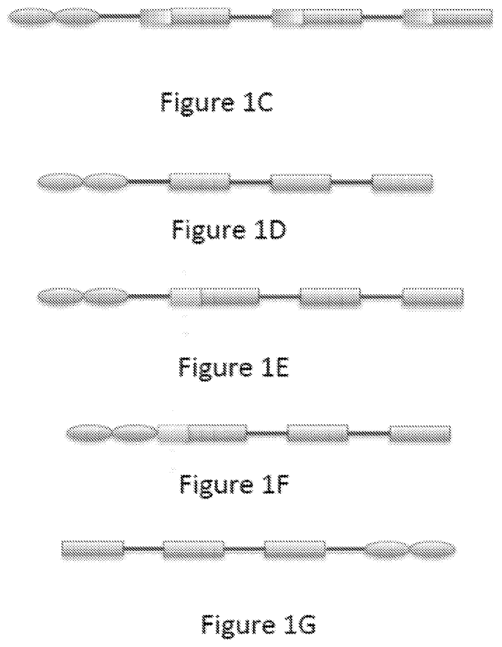

Full length human TNF-a has the sequence as set forth in FIG. 2 (SEQ ID NO:1). It is a type 2 transmembrane protein that is cleaved by the protease ADAMI7 to produce the cleaved, soluble TNF-a and uncleaved transmembrane TNF-a. Both soluble and transmembrane molecules signal through cognate receptors. Soluble TNF-a signals primarily through TNFR1, while transmembrane TNF-a signals primarily through TNFR2. The cleaved, soluble TNF-a has the sequence shown in SEQ ID NO:2. C-terminal to the cleavage site is a domain that forms the TNF-homology domain (THD), which is a sequence and structurally similar domain found in members of the TNF superfamily, that makes up the receptor binding domain of the molecule. Of note, a region N-terminal to the THD domain and including the ADAMI7 cleavage site is a domain of the molecule referred to as the “stalk region”. This stalk region does not appear to be found in the receptor-binding portion of the molecule. Accordingly, domains of TNF-a include from N- to C-terminus: N-terminal intracellular domain, a transmembrane domain, stalk region, ADAM 17 cleavage site within the stalk region and THD domain. The transmembrane domain terminates at amino acid 56. The stalk region is defined as amino acids 57-87 of the full-length sequence. The ADAMI 7 cleavage site is found between amino acids 76/77. The THD domain begins at amino acid 88 and extends to amino acid 233. This is summarized in FIG. 2.

Mutations in the THD have been identified that abrogate binding to TNFR1 and result in a molecule that agonizes TNFR2. The mutations are D143N and A145R, wherein the numbering is based on the sequence of soluble TNF-a. This corresponds to D219N and A221R wherein the numbering is based on the full length TNF-a sequence. That is, at these positions, the native sequences of D and A are mutated to an N and R, respectively. These mutations will be referred to as TNFR2 agonist sequences herein.

Accordingly, the present disclosure provides single-chain TNFR2 agonists (scTNFR2) comprising a first, second and third THD domain comprising the TNFR2 agonist sequences. In some embodiments all three of the THD domains of the scTNFR2 agonist comprise the TNFR2 agonist sequences. Sequence of the THD domain comprising the TNFR2 agonist sequences is found in SEQ ID NO:3.

The variants of this invention optionally include conservatively substituted Variants that apply to both amino acid and nucleic acid sequences. With respect to particular nucleic acid sequences, conservatively modified variants refer to those nucleic acids which encode identical or essentially identical amino acid sequences, or where the nucleic acid does not encode an amino acid sequence, to essentially identical sequences. Specifically, degenerate codon substitutions may be achieved by generating sequences in which the third position of one or more selected (or all) codons is substituted with mixed base and/or deoxyinosine residues (Batzer et al., Nucleic Acid Res. 19:5081 (1991); Ohtsuka et al., J. Biol. Chern. 260:2605-2608 (1985); Rossolini et al., Mol. Cell. Probes 8:91-98 (1994)). Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are silent variations, which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

With regard to conservative substitution of amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a conservatively modified variant where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following groups each contain amino acids that are conservative substitutions for one another:
1) Alanine (A), Glycine (G);
2) Serine (5), Threonine (T);
3) Aspartic acid (D), Glutamic acid (E);
4) Asparagine (N), Glutamine (Q);
5) Cysteine (C), Methionine (M);
6) Arginine (R), Lysine (K), Histidine (H);
7) Isoleucine (I), Leucine (L), Valine (V); and
8) Phenylalanine (F), Tyrosine (Y), Tryptophan (W).

### Dimerization Domains

One design choice is the nature of the dimerization domains of the fusion protein. Without being bound by theory, it is thought that dimerization enhances signaling by the TNFR2 agonist and also may improve half-life of the fusion protein. There are many different dimerization domains, such as Fc fusion proteins derived from other dimerizing molecules, such as IgE heavy chain domain 2 (EHD2) and IgM heavy chain domain 2 (MHD2). Fc protein moiety

The main therapeutic applications of Fc fusion proteins are (1) endowing the fusion partner protein with immunoglobulin Fc effector functions; or (2) increasing the circulating half-life of the fusion partner protein (Czajkowsky, et al., 2012, EMBO Mol Med. 4:1015-28). The primary effector functions of IgG proteins are Complement-Dependent Cytotoxicity (CDC) and Antibody-Dependent Cellular Cytotoxicity (ADCC), functions mediated by Fc binding to complement protein Clq and to IgG-Fc receptors (FcyR), respectively. These effector functions are important when the therapeutic protein is used to direct or enhance the immune response to a particular antigen target or cell. The fusion protein of this invention is designed solely to increase the circulating half-life of the TNFR2 Selective Agonist moiety, and effector functions are not needed and can even be toxic, and thus in some embodiments not desired. For instance, a scTNFR2 agonist-Fc fusion protein with an effector function-competent Fc can potentially kill the Treg cells that the fusion protein of this invention is seeking to activate and expand, exactly the opposite of the therapeutic goal for autoimmune diseases. There are four human IgG subclasses that differ in effector functions (CDC, ADCC), circulating half-life, and stability (Salfeld, J. G., 2007, Nature Biotechnology 25:1369-72). IgG1 possesses Fc effector functions, is the most abundant IgG subclass, and is the most commonly used subclass in US FDA-approved therapeutic proteins. IgG2 is deficient in Fc effector functions, but is subject to dimerization with other IgG2 molecules, and is also subject to instability due to scrambling of disulfide bonds in the hinge region. IgG3 possesses Fc effector functions, and has an extremely long, rigid hinge region. IgG4 is deficient in Fc effector functions, has a shorter circulating half-life than the other subclasses, and the IgG4 dimer is biochemically unstable due to only a single disulfide bond in the hinge region leading to the exchange of H chains between different IgG4 molecules. A skilled artisan would recognize that Fc protein moieties from IgG2 and IgG4 do not possess effector functions and can be used in this invention. The skilled artisan would also recognize that Fc sequence modifications have been described in the art that such that the hinge region of IgG2 Fc can be modified to prevent aggregation, or that the hinge region of IgG4 Fc can be modified to stabilize dimers. It will be appreciated by those of ordinary skill in the art that the IgG described in the sequences of the fusion constructs disclosed herein may be changed. That is, where an IgG1 sequence is disclosed, this can be exchanged with an IgG2 or IgG4 and the like.

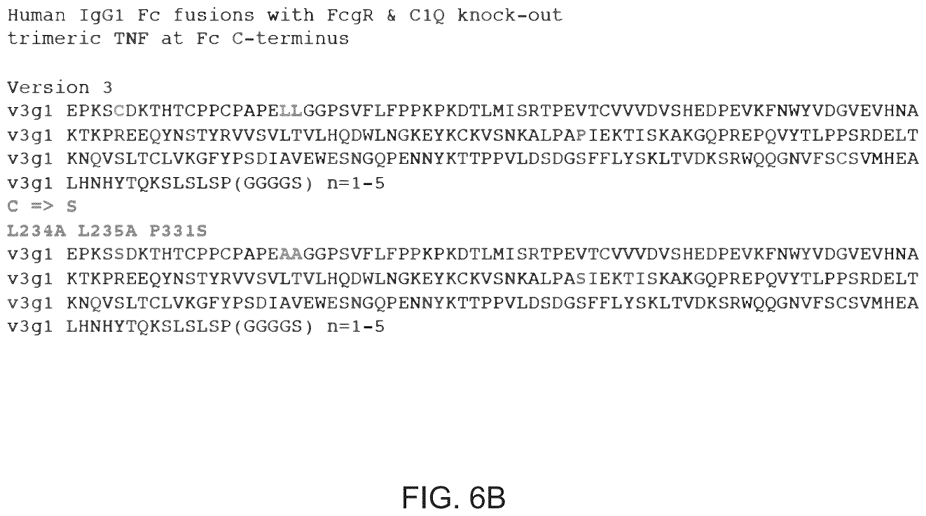

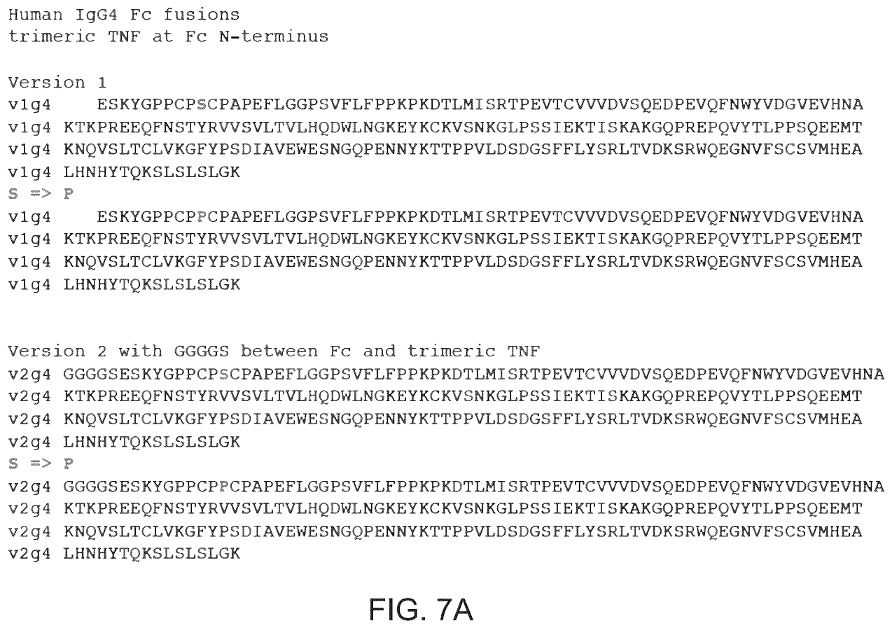

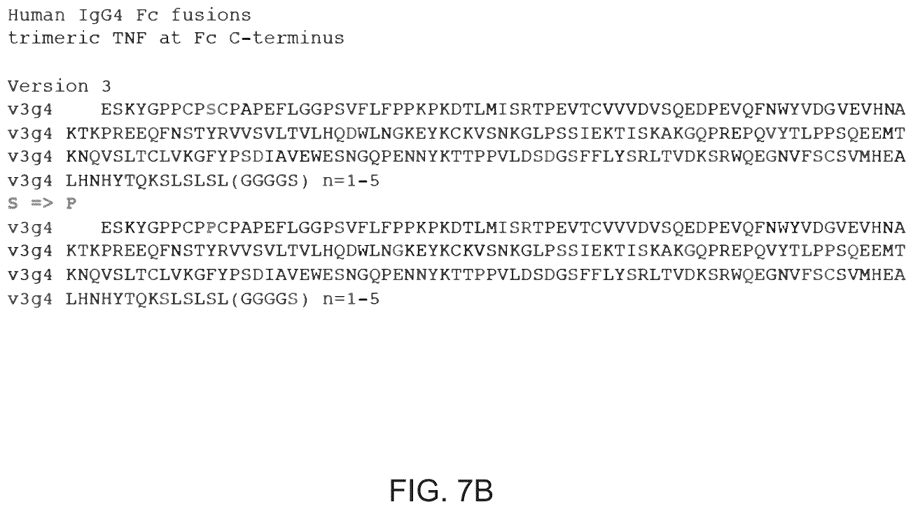

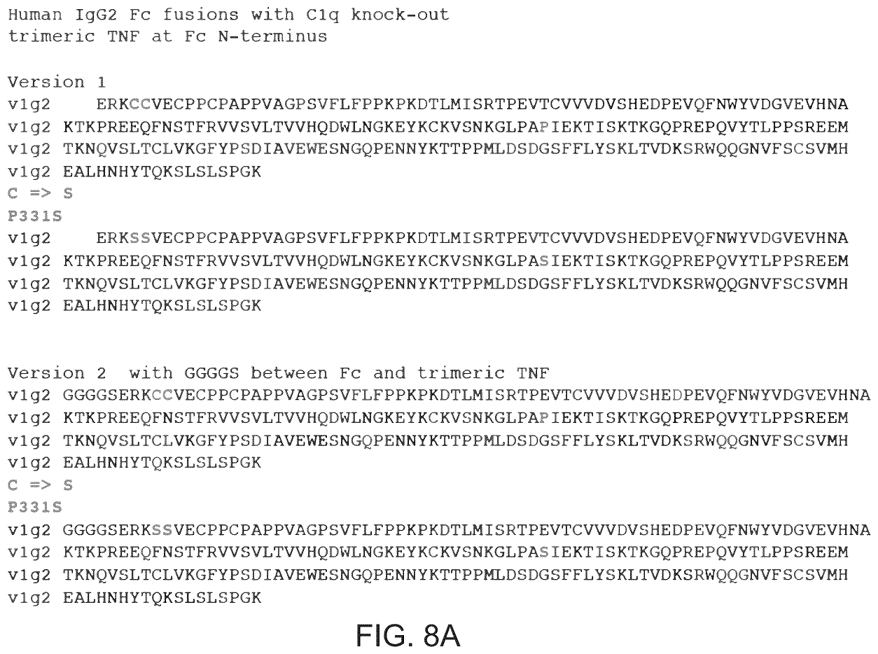

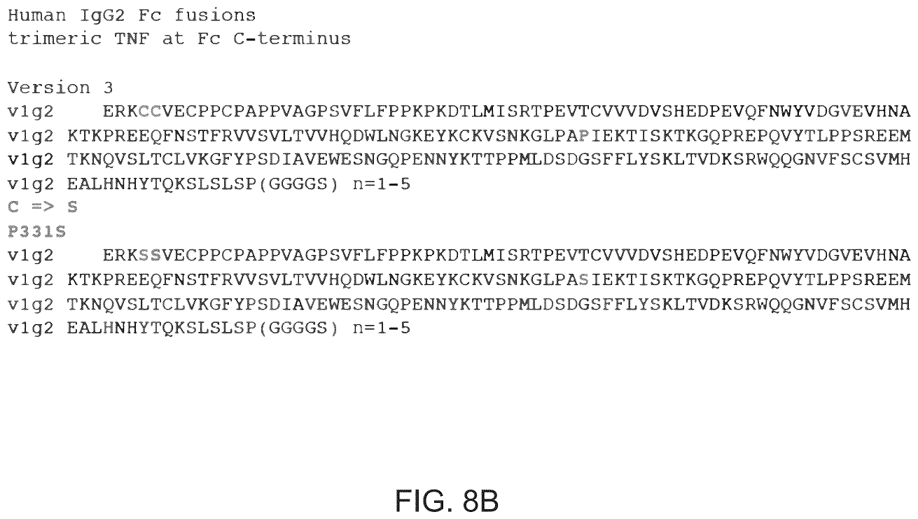

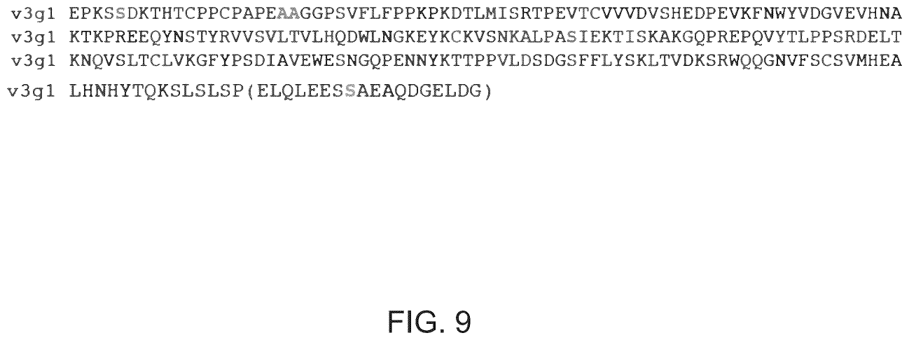

Alternatively, effector function-deficient variants of IgG1 have been generated. One such variant has an amino acid substitution at position N297, the location of an N-linked glycosylation site. Substitution of this asparagine residue removes the glycosylation site and significantly reduces ADCC and CDC activity (Tao, M. H., et al., 1989, J Immunol. 143:2595-2601). This variant is used as an exemplary case in the invention herein. Another effector function deficient IgG1 variant is IgG1 (L234F/L235E/P331S) (Oganesyan, et al., 2008, Acta Crystallogr D Biol Crystallogr. 64:700-4), which mutates amino acids in the Clq and FcyR binding sites, and one skilled in the art would consider using these or similar Fc variants to generate effector-deficient and stable scTNFR2 agonist-Fc fusion proteins. Other mutations at these sites, such as L234A and L235A can also be used in the fusion protein described herein. Exemplary IgG sequences and variants are shown in FIGS. 6A-6B, 7A-7B, and 8A-8B and in SEQ ID NOs: 5-22.

A skilled artisan would also recognize that forms of Fc protein moieties engineered to be stable monomers rather than dimers (Dumont, J. A., et., al., 2006, BioDrugs 20:151-60; Liu Z, et al., J Biol Chern. 2015 20; 290:7535-62) can also be combined with the TNFR2 selective agonist of this invention. In addition, a skilled artisan would recognize that a functionally monomeric heterodimer composed of a TNFR2 agonist-Fc H chain polypeptide combined with an Fc H chain polypeptide and assembled using bispecific antibody technology (Zhu Z, et al., 1997 Protein Sci. 6:781-8) can also be combined with the TNFR2 Selective Agonist of this invention. In addition, a skilled artisan will recognize that Fc variants that lack some or all of the hinge region can be used with this invention.

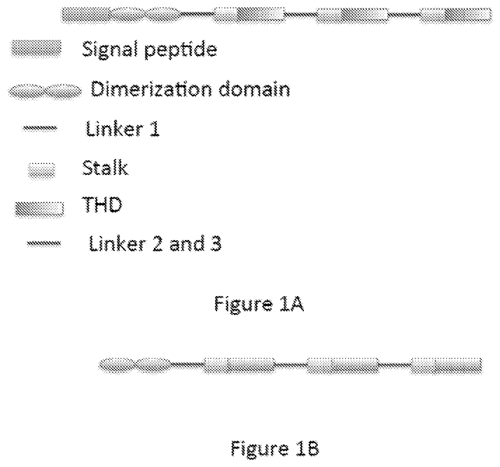

Fc fusion proteins can be made in two configurations, indicated here as X-Fc, where X, the scTNFR2 agonist fusion partner protein, is at the N-terminus and Fc is at the C-terminus, and Fc-X, where the Fc is at the N-terminus, and the scTNFR2 agonist fusion partner protein is at the C-terminus (FIGS. 1A-1G). There are examples in the literature showing that different fusion partners can have distinct preferences for N- or C-terminal Fc fusions. For instance, FGF21 has been shown to have a strong preference for the Fc-X configuration. Fc-FGF21 has receptor-activating bioactivity essentially the same as FGF21 itself, while FGF21-Fc has 1000-fold reduced bioactivity (Hecht, et al., 2012, PLOS One. 7 (1 1): e49345). A number of IL2 agonist Fc fusion proteins have been made for various applications, and these have been reported to have good IL-2 bioactivity when directly fused to Fc in both the Fc-X (Gillies, et al., 1992, Proc Natl Acad Sci, 89:1428-32; Bell, et al., 2015, J Autoimmun. 56:66-80) and X-Fc (Zheng, X. X., et al., 1999, J Immunol. 163:4041-8) configurations. Gavin, et al. (US20140286898 Al) describes Fc fusion proteins containing IL-2 and certain IL-2 variants in the in the Fc-X configuration that have bioactivity similar to that of the free IL-2 cytokine, but in contrast to the results of Zheng et al, (Zheng, X. X., et al., 1999, J Immunol. 1999, 163:4041-8) found that IL-2 variant fusion proteins in the X-Fc configuration have reduced or no bioactivity. Thus, whether an N-terminal dimerization domain or a C-terminal dimerization within any given fusion protein is preferred is unpredictable.

### EHD2

A recently described dimerization domain may also find use in connection with the scTNFR2 agonist described herein. This polypeptide was used to form dimers of other molecules in WO 2013/156148, which is expressly incorporated herein by reference. The EHD 2 sequence is:

(SEQ ID NO: 23)
DFTPPTVKILQSSCDGGGHFPPTIQLLCLVSGYTPGTINITWLED
GQVMDVDLSTASTTQEGELASTQSELTLSQKHWLSDRTYTCQVTY
QGHTFEDSTKKCADSN.

### MHD2

Another recently described dimerization domain may also find use in connection with the scTNFR2 agonist described herein. This polypeptide was used to form dimers of other molecules in WO 2013/156148. The MHD2 sequence is:

(SEQ ID NO: 24)
AELPPKVSVFVPPRDGFFGNPRKSKLICQATGFSPRQIQVSWLRE
GKQVGSGVTTDQVQAEAKESGPTTYKVTSTLTIKESDWLGQSMFT
CRVDHRGLTFQQNASSMCVPD.

### Linker

The amino acid sequence at the junction between the Fc and the fusion partner protein can be either (1) a direct fusion of the two protein sequences or (2) a fusion with an intervening linker peptide. Of the 10 Fc fusion proteins that are presently approved by the LIS FDA for clinical use (TABLE I), 8 are direct fusions of the fusion partner protein with Fc, while 2 possess linker peptides, so many Fc fusion proteins can be functional without linker peptides. Linker peptides are included as spacers between the two protein moieties. Linker peptides can promote proper protein folding and stability of the component protein moieties, improve protein expression, and enable better bioactivity of the component protein moieties (Chen, et al., 2013, Adv Drug Deliv Rev. 65:1357-69). Peptide linkers used in many fusion proteins are designed to be unstructured flexible peptides. A study of the length, sequence, and conformation of linkers peptides between independent structural domains in natural proteins has provided a theoretical basis for the design of flexible peptide linkers (Argos, 1990, J Mol Biol. 211:943-5 8). Argos provided the guidance that long flexible linker peptides be composed of small nonpolar residues like Glycine and small polar resides like Serine and Threonine, with multiple Glycine residues enabling a highly flexible conformation and Serine or Threonine providing polar surface area to limit hydrophobic interaction within the peptide or with the component fusion protein moieties. Many peptide linkers described in the literature are rich in glycine and serine, such as repeats of the sequence GGGGS (SEQ ID NO:25), although an artisan skilled in the art will recognize that other sequences following the general recommendations of Argos (Argos, 1990, J Mol Biol. 20; 211 (4): 943-58) can also be used. In some embodiments polypeptide sequences from one of the fusion partners may be used as a linker. For instance, N- or C-terminal extensions from TNF-a or a dimerization domain, such as Fc, could be used all or part of the linker between the fusion partners. In some embodiments the C-terminal extension from human IgG finds use as a linker and is shown as: ELQLEESSAEAQDGELDG (SEQ ID NO:41) or a variant of this also finds use as a linker: ELQLEESSAEAQGG (SEQ ID NO:42).

TABLE I
US FDA-approved Fc fusion proteins and their characteristics

| | FC | Fusion | N vs C | Linker | Half-life |
|---|---|---|---|---|---|
| Drug | Isotype | Partner | fusion | Peptide | (days) |
| Romiplostim | G1 | TPO-R peptide | C | Y | 3.5 |
| Etanercept | G1 | P75 TNFa-R | N | N | 4.3 |
| Alefacept | G1 | LFA3 | N | N | 10.1 |
| Rilonacept | G1 | IL1-R | N | N | 8.6 |
| Abatacept | G1 | CTLA4 | N | N | 16.7 |
| Belatacept | G1 | CTLA4 (mut) | N | N | 9.8 |
| Aflibercept | G1 | VEGF RI + R2 | N | N | n/a |
| Dulaglutide | G4 (mut) | GLP1 | N | Y | 3.7 |
| Electate | G1 | FVIII | N | N | 0.8 |
| Alprolix | G1 | FIX | N | N | 3.6 |

In some embodiments, particularly when the fusion protein is in the DD-X configuration, the dimerization domain (DD), i.e. Fc, is directly linked to the N-terminus of the single-chain THD, i.e. TNFR2 agonist.

In some embodiments, particularly when the fusion protein is in the DD-X configuration, the linker between the N-terminus of the first THD domain of the scTNFR2 agonist is sequence from TNF-a itself. That is, sequences from the native TNF-a stalk region are used as a linker between the THD domain of the TNFR2 agonist and the C-terminus of the Fc domain. The linker between the THD domain of the TNFR2 agonist and the C-terminus of the Fc domain contains from 1 to 31 amino acids or contains 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 amino acids. The stalk region contains the sequences shown below and the linker using contiguous amino acids from this region may include from 1 to 31 contiguous amino acids of this sequence. I he sequence from the first amino acid of the stalk region to last amino acid prior to the THD domain includes: GPQREEFPRDLSLISPLAQA VRSSSRTPSDK (SEQ ID NO: 26). In some embodiments sequences comprising at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 contiguous sequences from the stalk region can be used as a linker between the N-terminus of the scTNF agonist and dimerization domain.

In some embodiments, other linkers, such as combinations of Gly and Ser find use as linkers. In some embodiments linkers using (GGGGS) n, where n=1-5 (SEQ ID NO: 44) find use as linkers between the dimerization domain, i.e. Fc and first THD of the scTNFR2 agonist. In some embodiments, combinations of the stalk region sequences and Gly/Ser amino acids find use as the linker.

In some embodiments a linker peptide of 5, 10, 15, or 20 amino acids will have a maximum fully extended length of 17.5 A, 35 A, 52.5 A, or 70 A, respectively. The maximal end-to-end length of the peptide linker can also be a guide for defining the characteristics of a peptide linker in this invention. The goal of a linker peptide within the current invention is to enable attainment of an appropriate conformation and orientation of the individual fusion protein moieties to allow the engagement of the TNFR2 Selective Agonist moiety with its cognate receptor and allow the binding of the Fc moiety to the FcRn to enable fusion protein recycling and a prolonged circulating half-life. Since the factors influencing these interactions are difficult to predict, the requirement for and the proper length of a linker peptide must be empirically tested and determined. Many Fc fusion proteins do not require linker peptides, as evidenced by the 8 out of 10 US FDA-approved Fc fusion proteins lacking such peptides listed in Table I. In contrast, Dulaglutide, a fusion of GLP-1 and Fc, contains a 15 residue peptide linker which has a strong influence on bioactivity (Glaesner, U.S. Pat. No. 7,452,966 B2).

In the context of the single-chain TNFR2 agonist, other linkers may be found between the THD domains. That is, a linker may be found between the first and second and then the second third THD domain of the TNFR2 agonist. The linkers may be the same or may be different. In some embodiments the linkers may be any linker outlined herein including GGGGS (SEQ ID NO: 25) linkers. In some embodiments the linker may comprise multiple units of the GGGGS (SEQ ID NO: 25) sequence as described as (GGGGS)ₙ, wherein n=1, 2, 3, 4, 5, 6, 7, 8, 9 or 10 (SEQ ID NO: 45). In some embodiments sequences from the stalk region find use as linkers between the THDs. In addition, in some embodiments, combinations of Gly/Ser amino acids as well as contiguous amino acids from the stalk region find use as linkers between THDs. Linker between the first and second THDs may be the same or different from the linker between the second and third THD but generally both linkers will be comprised of (GGGGS) n, wherein n=1, 2, 3, 4, 5, 6, 7, 8, 9 or 10 (SEQ ID NO: 45) and/or contiguous sequences from the stalk region.

In other embodiments, particularly in the X-DD configuration, a linker may be placed between the C-terminus of the third THD domain and the N-terminus of dimerization domain, i.e. Fc domain. Again this can be Gly/Ser linkers as described herein and may comprise (GGGGS) n, where n=1-5 (SEQ ID NO: 44).

### Fusion Proteins

Accordingly, the present disclosure provides scTNFR2 fusion proteins comprising a dimerization domain, three THD's each comprising the D143N/A145R mutations to confer selectivity for TNFR2, and a linker between each of the THDs. In some embodiments the dimerization domain is at the N-terminus of the scTNFR2 agonist domain, while in other embodiments the dimerization domain as at the C-terminus of the molecule.

Fusion proteins disclosed herein comprise the following formulas: DD-L1-THD-L2-THD-L3-THD or THD-L2-THD-L3-THD-L1-DD, where DD is a dimerization domain as described herein. Dimerization domains are selected from IgG1, IgG2 an IgG4 Fc domains lacking effector function. In one embodiment the Fc is from IgG2 and lacking effector function. In one embodiment the Fc is from IgG4. In one embodiment the dimerization domain is EHD2 or MHD2. Then the dimerization domain is at the N-terminus of the scTNFR2 agonist protein, the linker (LI) is preferably (GGGGS), where n=1-5 (SEQ ID NO: 44), although in some embodiments the LI linker comprises some or all of the stalk region from TNF-a. All fusion proteins of the invention disclosed herein contain THD with the TNFR2 agonist selective sequences D143N/A145R and are referred to below as THD. Linkers (L2 and L3) between the first and second, and second and third THD may also be constructed from GGGGS (SEQ ID NO: 25), G/S linkers or from some or all of the stalk region. When the dimerization domain is at the C-terminus of the scTNFR2 agonist protein there may not be a linker, or the linker may comprise (GGGGS), where n=1-5 (SEQ ID NO: 44). Preferred configurations of fusion proteins include:
DD-L1-THD-L2-THD-L3-THD, wherein DD is the IgG4 Fc with mutation(s) eliminating effector function, LI, L2 and L3 are GGGGS (SEQ ID NO: 25);
DD-L1-THD-L2-THD-L3-THD, wherein DD is the IgG4 Fc with mutation(s) eliminating effector function, LI is GGGGSGGGGS (SEQ ID NO:27), L2 and L3 are both GGGGS (SEQ ID NO: 25);
DD-L1-THD-L2-THD-L3-THD, wherein DD is the IgG1 Fc with mutation(s) eliminating effector function, LI is VRSSSRTPSDK (SEQ ID NO: 4), L2 and L3 are both GGGGS (SEQ ID NO: 25);
DD-L1-THD-L2-THD-L3-THD, wherein DD is the IgG1 Fc with mutation(s) eliminating effector function, LI is VRSSSRTPSDK (SEQ ID NO:4), L2 and L3 are both SSRTPSDK (SEQ ID NO: 28);
DD-L1-THD-L2-THD-L3-THD, wherein DD is the IgG1 Fc with mutation(s) eliminating effector function, LI is GPQREEFPRDLSLISPLAQAVRSSSRTPSDK (SEQ ID NO: 26), L2 and L3 are both GGGGS (SEQ ID NO: 25);
DD-L1-THD-L2-THD-L3-THD, wherein DD is the IgG1 Fc with mutation(s) eliminating effector function, LI is GPQREEFPRDLSLISPLAQAVRSSSRTPSDK (SEQ ID NO: 26), L2 and L3 are both SSRTPSDK (SEQ ID NO:28);
DD-L1-THD-L2-THD-L3-THD, wherein DD is the IgG1 Fc with mutation(s) eliminating effector function, LI is GPQREEFPRDLSLISPLAQAVRSSSRTPSDK (SEQ ID NO: 26), L2 and L3 are both VRSSSRTPSDK (SEQ ID NO: 4);
DD-L1-THD-L2-THD-L3-THD, wherein DD is the IgG1 Fc with mutation(s) eliminating effector function, LI is GGGGSVRSSSRTPSDK (SEQ ID NO:29), L2 and L3 are both VRSSSRTPSDK (SEQ ID NO: 4);
DD-L1-THD-L2-THD-L3-THD, wherein DD is the IgG1 Fc with mutation(s) eliminating effector function, LI is GGGGSVRSSSRTPSDK (SEQ ID NO: 29), L2 and L3 are both GGGGSSSRTPSDK (SEQ ID NQ: 30);
DD-L1-THD-L2-THD-L3-THD, wherein DD is the IgG1 Fc with mutations eliminating effector function, LI, L2 and L3 are GGGGS (SEQ ID NO: 25);
DD-L1-THD-L2-THD-L3-THD, wherein DD is the IgG1 Fc with mutations eliminating effector function, LI is GGGGSGGGGS (SEQ ID NO:27), L2 and L3 arc both GGGGS (SEQ ID NO: 25);
DD-L1-THD-L2-THD-L3-THD, wherein DD is the IgG1 Fc with mutations eliminating effector function, LI is VRSSSRTPSDK (SEQ ID NO: 4), L2 and L3 are both GGGGS (SEQ ID NO: 25);
DD-L1-THD-L2-THD-L3-THD, wherein DD is the IgG1 Fc with mutations eliminating effector function, LI is VRSSSRTPSDK (SEQ ID NO:4), L2 and L3 are both SSRTPSDK (SEQ ID NO: 28);
DD-L1-THD-L2-THD-L3-THD, wherein DD is the IgG1 Fc with mutations eliminating effector function, LI is GPQREEFPRDLSLISPLAQAVRSSSRTPSDK (SEQ ID NO: 26), L2 and L3 are both GGGGS (SEQ ID NO: 25);
DD-L1-THD-L2-THD-L3-THD, wherein DD is the IgG1 Fc with mutations eliminating effector function, LI is GPQREEFPRDLSLISPLAQAVRSSSRTPSDK (SEQ ID NO: 26), L2 and L3 are both SSRTPSDK (SEQ ID NO: 28);
DD-L1-THD-L2-THD-L3-THD, wherein DD is the IgG1 Fc with mutations eliminating effector function, LI is GPQREEFPRDLSLISPLAQAVRSSSRTPSDK (SEQ ID NO: 26), L2 and L3 are both VRSSSRTPSDK (SEQ ID NO: 4);
DD-L1-THD-L2-THD-L3-THD, wherein DD is the IgG1 Fc with mutations eliminating effector function, LI is GGGGSVRSSSRTPSDK (SEQ ID NO:29), L2 and L3 are both VRSSSRTPSDK (SEQ ID NO: 4);
DD-L1-THD-L2-TJID-L3-THD, wherein DD is the IgG1 Fc with mutations eliminating effector function, LI is GGGGSVRSSSRTPSDK (SEQ ID NO: 29), L2 and L3 are both GGGGSSSRTPSDK (SEQ ID NO:30);
DD-L1-THD-L2-THD-L3-THD, wherein DD is the IgG4 Fc, LI, L2 and L3 are GGGGS (SEQ ID NO: 25);
DD-L1-THD-L2-THD-L3-THD, wherein DD is the IgG4 Fc, LI is GGGGSGGGGS (SEQ ID NO: 27), L2 and L3 are both GGGGS (SEQ ID NO: 25);
DD-L1-THD-L2-THD-L3-THD, wherein DD is the IgG4 Fc, LI is VRSSSRTPSDK (SEQ ID NO: 4), L2 and L3 are both GGGGS (SEQ ID NO: 25);
DD-L1-THD-L2-THD-L3-THD, wherein DD is the IgG4 Fc, LI is VRSSSRTPSDK (SEQ ID NO: 4), L2 and L3 are both SSRTPSDK (SEQ ID NO: 28);
DD-L1-THD-L2-THD-L3-THD, wherein DD is the IgG4 Fc, LI is GPQREEFPRDLSLISPLAQAVRSSSRTPSDK (SEQ ID NO: 26), L2 and L3 are both GGGGS (SEQ ID NO: 25);
DD-L1-THD-L2-THD-L3-THD, wherein DD is the IgG4 Fc, LI is GPQREEFPRDLSLISPLAQAVRSSSRTPSDK (SEQ ID NO: 26), L2 and L3 are both SSRTPSDK (SEQ ID NO: 28);
DD-L1-THD-L2-THD-L3-THD, wherein DD is the IgG4 Fc, LI is GPQREEFPRDLSLISPLAQAVRSSSRTPSDK (SEQ ID NO: 26), L2 and L3 are both VRSSSRTPSDK (SEQ ID NO: 4);
DD-L1-THD-L2-THD-L3-THD, wherein DD is the, LI is GGGGSVRSSSRTPSDK (SEQ ID NO: 29), L2 and L3 are both VRSSSRTPSDK (SEQ ID NO: 4);
DD-L1-THD-L2-THD-L3-THD, wherein DD is the lgG4 Fc, LI is GGGGSVRSSSRTPSDK (SEQ ID NO: 29), L2 and L3 are both GGGGSSSRTPSDK (SEQ ID NO: 30);
THD-L2-THD-L3-THD-L1-DD, wherein DD is the IgG1 Fc with mutations eliminating effector function, LI, L2 and L3 are GGGGS (SEQ ID NO: 25);
THD-L2-THD-L3-THD-L1-DD, wherein DD is the IgG1 Fc with mutations eliminating effector function, LI is GGGGS (SEQ ID NO: 25) and L2 and L3 are SSRTPSDK (SEQ ID NO: 28);
THD-L2-THD-L3-THD-DD, wherein DD is the IgG1 Fc with mutations eliminating effector function, L2 and L3 are GGGGS (SEQ ID NO: 25) and scTNFR2 agonist domain is fused directly to DD;
THD-L2-THD-L3-THD-DD, wherein DD is the IgG1 Fc with mutations eliminating effector function, L2 and L3 are SSRTPSDK (SEQ ID NO: 28) and scTNFR2 agonist domain is fused directly to DD;
THD-L2-THD-L3-THD-DD, wherein DD is the IgG1 Fc with mutations eliminating effector function, L2 and L3 are GGGGSSSRTPSDK (SEQ ID NO: 30) and scTNFR2 agonist domain is fused directly to DD;
THD-L2-THD-L3-THD-DD, wherein DD is the IgG1 Fc with mutations eliminating effector function, L2 and L3 are VRSSSRTPSDK (SEQ ID NO: 4) and scTNFR2 agonist domain is fused directly to DD;
THD-L2-THD-L3-THD-L1-DD, wherein DD is the IgG4 Fc, LI, L2 and L3 are GGGGS (SEQ ID NO: 25);
THD-L2-THD-L3-THD-L1-DD, wherein DD is the IgG4 Fc, LI is GGGGS (SEQ ID NO: 25) and L2 and L3 are SSRTPSDK (SEQ ID NO: 28);
THD-L2-THD-L3-THD-DD, wherein DD is the IgG4 Fc, L2 and L3 are GGGGS (SEQ ID NO: 25) and scTNFR2 agonist domain is fused directly to DD;
THD-L2-THD-L3-THD-DD, wherein DD is the IgG4 Fc, L2 and L3 are SSRTPSDK (SEQ ID NO: 28) and scTNFR2 agonist domain is fused directly to DD;
THD-L2-THD-L3-THD-DD, wherein DD is the IgG4, L2 and L3 are GGGGSSSRTPSDK (SEQ ID NO: 30) and scTNFR2 agonist domain is fused directly to DD;
THD-L2-THD-L3-THD-DD, wherein DD is the IgG4 Fc, L2 and L3 are VRSSSRTPSDK (SEQ ID NO: 4) and scTNFR2 agonist domain is fused directly to DD;
THD-L2-THD-L3-THD-L1-DD, wherein DD is the IgG4 Fc with mutation(s) eliminating effector function, LI, L2 and L3 are GGGGS (SEQ ID NO: 25);
THD-L2-THD-L3-THD-L1-DD, wherein DD is the IgG4 Fc with mutation(s) eliminating effector function, LI is GGGGS (SEQ ID NO: 25) and L2 and L3 are SSRTPSDK (SEQ ID NO: 28);
THD-L2-THD-L3-THD-DD, wherein DD is the IgG4 Fc with mutation(s) eliminating effector function, L2 and L3 are GGGGS (SEQ ID NO: 25) and scTNFR2 agonist domain is fused directly to DD;
THD-L2-THD-L3-THD-DD, wherein DD is the IgG4 Fc with mutations eliminating effector function, L2 and L3 are SSRTPSDK (SEQ ID NO: 28) and scTNFR2 agonist domain is fused directly to DD;
THD-L2-THD-L3-THD-DD, wherein DD is the IgG4 Fc with mutations eliminating effector function, L2 and L3 are GGGGSSSRTPSDK (SEQ ID NO: 30) and scTNFR2 agonist domain is fused directly to DD;
THD-L2-THD-L3-THD-DD, wherein DD is the IgG4 Fc with mutations eliminating effector function, L2 and L3 are VRSSSRTPSDK (SEQ ID NO: 4) and scTNFR2 agonist domain is fused directly to DD.

In some embodiments, the Fc-scTNFR2 agonist fusion protein comprises the sequence as shown in SEQ ID NO:31, 32, 34, or 35. In some embodiments the Fc-scTNFR2 agonist fusion protein comprises the sequence as shown in SEQ ID NOs: 101, 102, 103, 104, 105, 106, 107, 108, 109, 110, 111, 112, 118, 119 or 120. In some embodiments the scTNFR2 agonist fusion protein comprises a protein having at least 80% or 85% or 90% or 95% or 96% or 97% or 98% or 99% identity with SEQ ID NO:31, 32, 34 or 35 or SEQ IS NOs: 101, 102, 103, 104, 105, 106, 107, 108, 109, 110, 111, 112, 118, 119 or 120. In some embodiments, the present disclosure provides a nucleic acid encoding a protein as set forth in SEQ ID NO:31, 32, 34, or 35 or a protein having at least 80% or 85% or 90% or 95% or 96% or 97% or 98% or 99% identity with SEQ ID NO:31, 32, 34 or 35 or SEQ ID NOS 101, 102, 103, 104, 105, 106, 107, 108, 109, 110, 111, 112, 118, 119 or 120. In some embodiments the nucleic acid comprises a nucleic acid sequence having at least 80% or 85% or 90% or 95% or 96% or 97% or 98% or 99% identity with SEQ ID NO:36, 37, 38 or 39. In some embodiments the nucleic acid comprises the sequence shown in SEQ ID NO: 36, 27, 28 or 39.

### Bioassays

Robust and quantitative bioassays are necessary for the characterization of the biological activity of candidate proteins. These assays should measure the activation of the TNFR2 30 receptor, measure the downstream functional consequences of activation in Tregs, and measure therapeutically-relevant outcomes and functions of the activated Tregs. These assays can be used the measure the therapeutic activity and potency of scTNFR2 Selective Agonist molecules, and can also be used for measurement of the pharmacodynamics of an scTNFR2 Selective Agonist in animals or in humans. One assay measures the TNF-a mediated caspase activity. In cells lacking TNFR1 or when TNFR1 cannot signal, this is a measure of TNFR2 activation. Another assay for functional activation measures TNFR2 agonist stimulated proliferation of Treg cells. One of ordinary skill in the art will recognize that Treg proliferation can be measured by tritiated thymidine incorporation into purified Treg cells, by an increase in Treg cell numbers in a mixed population of cells measured by flow cytometry and the frequencies of CD4+CD25+FOXP3+ or the CD4+CD25+CD127− marker phenotypes, by increased expression in Treg cells of proliferation-associated cell cycle proteins, such as Ki-67, or by measurement of the cell division-associated dilution of a vital fluorescent dye such as carboxyfluorescein succinimidyl ester (CFSE) by flow cytometry in Treg cells. Accordingly, in some embodiments the present disclosure provides methods of stimulating or expanding Tregs. In some embodiments the fusion proteins of described herein stimulate the expansion of Tregs more potently that EHD2-TNFR2 agonist (disclosed in Dong et al. PNAS 2016).

Other assays include the Kym-1 cell viability assay disclosed in the examples. In some embodiments the disclosure provides Fc-TNFR2 agonist fusion proteins that reduce viability of Kym-1 cells following culture as descrived herein. In some embodiments the Fc-TNFR2 agonists reduce the viability of Kym-1 cells more than EHD2-TNFR2 agonist (disclosed in Dong et al. PNAS 2016).

### Formulation

Pharmaceutical compositions of the fusion proteins of the present invention are defined as formulated for parenteral (particularly intravenous or subcutaneous) delivery according to conventional methods. In general, pharmaceutical formulations will include fusion proteins of the present invention in combination with a pharmaceutically acceptable vehicle, such as saline, buffered saline, 5% dextrose in water, or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19.sup.th ed., 1995.

As an illustration, pharmaceutical formulations may be supplied as a kit comprising a container that comprises fusion proteins of the present invention. Therapeutic proteins can be provided in the form of an injectable solution for single or multiple doses, as a sterile powder that will be reconstituted before injection, or as a prefilled syringe. Such a kit may further comprise written information on indications and usage of the pharmaceutical composition. Moreover, such information may include a statement that the fusion proteins of the present invention is contraindicated in patients with known hypersensitivity to fusion proteins of the present invention.

The scTNFR2 selective agonist fusion proteins of this invention can be incorporated into compositions, including pharmaceutical compositions. Such compositions typically include the protein and a pharmaceutically acceptable carrier. As used herein, the term “pharmaceutically acceptable carrier” includes, but is not limited to, saline, solvents, dispersion media, coatings, antibacterial and antifungal agents isotonic and absorption delaying agents, and the like, compatible with pharmaceutical administration. Supplementary active compounds (e.g., antibiotics) can also be incorporated into the compositions.

A pharmaceutical composition is formulated to be compatible with its intended route of administration. The scTNFR2 selective agonist fusion proteins of the invention is likely that to be administered through a parenteral route. Examples of parenteral routes of administration include, for example, intravenous, intradermal, and subcutaneous. Solutions or suspensions used for parenteral application can include the following components: a sterile diluent such as water for injection, saline solution, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as mono- and/or dibasic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The maintenance of the required particle size in the case of dispersion may be facilitated by the use of surfactants, e.g., Polysorbate or Tween. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In one embodiment, the scTNFR2 selective agonist fusion protein is prepared with carriers that will protect the scTNFR2 selective agonist fusion protein against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques.

The pharmaceutical compositions can be included in a container, pack, or dispenser together with instructions for administration.

### Administration

Fusion proteins of the present invention will preferably be administered by the parenteral route. The subcutaneous route is the preferred route, but intravenous, intramuscular, and subdermal administration can also be used. For subcutaneous or intramuscular routes, depots and depot formulations can be used. For certain diseases specialized routes of administration can be used. For instance, for eye diseases, such as but not limited to optic neuritis, intraocular injection can be used. Fusion proteins can be used in a concentration of about 0.1 to 10 mcg/ml of total volume, although concentrations in the range of 0.01 mcg/ml to 100 mcg/ml may be used. In some embodiments peripheral administration is used to treat neurological disorders. In some embodiments intrathecal administration is used, which can deliver the fusion proteins into the spinal fluid which can bypass the blood brain barrier.

Determination of dose is within the level of ordinary skill in the art. Dosing is daily or weekly over the period of treatment, or may be at another intermittent frequency. Intravenous administration will be by bolus injection or infusion over a typical period of one to several hours. Sustained release formulations can also be employed. In general, a therapeutically effective amount of fusion proteins of the present invention is an amount sufficient to produce a clinically significant change in the treated condition, such as a clinically significant change in circulating Treg cells, a clinically significant change in Treg cells present within a diseased tissue, or a clinically significant change in a disease symptom.

The data obtained from the cell culture assays and animal studies can be used in formulating a range of dosage for use in humans. The dosage of such compounds lies preferably within a range of circulating concentrations that include the half maximal effective concentration (EC50; i.e., the concentration of the test compound which achieves a half-maximal stimulation of Treg cells) with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 as determined in cell culture. Such information can be used to more accurately determine useful doses in humans. Levels in plasma may be measured, for example, by enzyme-linked immunosorbent assays.

As defined herein, a therapeutically effective amount of a scTNFR2 selective agonist fusion protein (i.e., an effective dosage) depends on the polypeptide selected and the dose frequency. For instance, single dose amounts in the range of approximately 0.01 to 50 mg/kg of patient body weight can be administered; in some embodiments, about 0.05 to 10 mg/kg, or 0.1 to 25 mg/kg of patient body weight can be administered; in some embodiments about 0.5 to 10 mg/kg of patient body weight can be administered. In some embodiments about 0.1 mg/kg, 0.5 mg/kg, 1 mg/kg, 2.5 mg/kg, 5 mg/kg, 7.5 mg/kg, 10 mg/kg, or 20 mg/kg or 40 mg/kg or 50 mg/kg of patient body can be administered. In some embodiments, for instance when intraocular administration is used, the concentration per patient body weight is in appropriate measure to use. Rather, a total of 0.5 mg, or 1 mg or 1.5 mg or 2 mg or 2.5 mg or 3 mg or 3.5 mg or 4 mg or 5 mg of fusion protein are administered in each eye. The compositions can be administered from one time per day to one or more times per week, or one or more times per month; including once every other day, or twice a week or twice a month. The skilled artisan will appreciate that certain factors may influence the dosage and timing required to effectively treat a subject, including but not limited to the severity of the disease or disorder, previous treatments, the general health and/or age of the subject, the level of Treg cells present in the patient, and other diseases present. Moreover, treatment of a subject with a therapeutically effective amount of the TNFR2 selective agonist fusion protein of the invention is likely to be a series of treatments.

### Diseases

Some of the diseases that can benefit from the therapy of this invention have been noted. However, the role of Treg cells in autoimmune diseases is a very active area of research, and additional diseases will likely be identified as treatable by this invention. Autoimmune diseases are defined as human diseases in which the immune system attacks its own proteins, cells, and tissues. A comprehensive listing and review of autoimmune diseases can be found in The Autoimmune Diseases (Rose and Mackay, 2014, Academic Press).

As disclosed herein, even when administered peripherally, scTNFR2 agonist proteins may be used to treat neurological disorders, particularly those characterized by elevated TNF-a. In one embodiment the scTNFR2 molecules disclosed herein find use in treating neurological disorders, e.g., by reducing inflammation in the brain, protecting myelination of neurons and/or promoting remyelination of neurons. Accordingly, neurological disorders particularly amenable to the methods disclosed herein include art-recognized inflammatory neurodegenerative diseases, which may result in the destruction 36 of myelin or may include other neurological disorders not necessarily characterized by myelin destruction but are characterized by elevated levels of TNF-α.

In one embodiment, neurodegenerative diseases are a group of diseases typified by deterioration of neurons and/or their myelin sheath. This destruction of neurons eventually leads to dysfunction and disabilities. Often inflammation, thought to be mediated by microglial cells, is found to be a component of neurodegenerative diseases and adds to the pathogenesis of the neurodegeneration. Collectively, these diseases comprise the art-recognized neurodegenerative diseases. Neuro inflammation may occur years prior to any considerable loss of neurons in some neurodegenerative disorders. For example, 70% of dopaminergic neurons are lost from the substantia nigra before patients begin to manifest the clinical signs of Parkinson's disease, see, e.g., Factor and Weiner (2008) Parkinson's Disease: Diagnosis and Clinical Management. Many different types of immune cells, including macrophages, neutrophils, T cells, astrocytes, and microglia, can contributed to the pathology of immune-related diseases, like Multiple Sclerosis (M.S.), Parkinson's disease, Huntington's disease, dementia (including but not exclusively diseases like Alzheimer's disease, frontotemporal dementia, trauma related dementia (punch drunk), HIV-associated and Lewy Body dementia), amyotrophic lateral sclerosis (ALS), prion diseases, etc. More specifically, in MS the injury to myelin is mediated by an inflammatory response and M.S. Pathogenesis is exacerbated when leukocytes infiltrate the CNS.

Accordingly, neurodegenerative diseases include but are not limited to: multiple sclerosis (MS), Optic Neuritis, Parkinson's disease, amyloidosis (e.g., Alzheimer's disease), amyotrophic lateral sclerosis (ALS), HIV-associated dementia, stroke/cerebral ischemia, head trauma, spinal cord injury, Huntington's disease, migraine, cerebral amyloid angiopathy, AIDS, age-related cognitive decline; mild cognitive impairment and prion diseases in a mammal, and preferably in a human.

Multiple sclerosis (MS) is a chronic inflammatory neurodegenerative disease of the central nervous system (CNS) that affects approximately 1,100,000 people all over the world, in particular affects young adults. MS is characterized pathologically by demyelination of neural tissue, which results clinically in one of many forms of the disease, ranging from benign to chronic-progressive patterns of the disease state. More specifically, five main forms of multiple sclerosis have been described: 1) benign multiple sclerosis: 2) relapsing-remitting multiple sclerosis (RRMS); 3) secondary progressive multiple sclerosis (SPMS); 4) primary progressive multiple sclerosis (PPMS); and 5) progressive-relapsing multiple sclerosis (PRMS). Chronic progressive multiple sclerosis is a term used to collectively refer to SPMS, PPMS, and PRMS. The relapsing forms of multiple sclerosis are SPMS with superimposed relapses, RRMS and PRMS.

Throughout the course of the disease there is a progressive destruction of the myelin sheath surrounding axons. Since intact myelin is essential in the preservation of axonal integrity systematic destruction eventually leads, clinically, to various neurological dysfunctions including numbness and pain, problems with coordination and balance, blindness, and general cognitive impairment.

Parkinson's disease, another inflammatory neurodegenerative disease, is characterized by movement disorders, including muscle rigidity and slow physical movements.

Amyloidosis develops when certain proteins have altered structure and tend to bind to each building up in particular tissue and blocking the normal tissue functioning. These altered structured proteins are called amyloids. Often amyloidoses is split into two categories: primary or secondary. Primary amyloidoses occur from an illness with improper immune cell function. Secondary amyloidoses usually arise from a complication of some other chronic infectious or inflammatory diseases. Examples of such include Alzheimer's disease and rheumatoid arthritis. The underlying problem in secondary amyloidosis is inflammation.

Alzheimer's disease is another type of inflammatory neurodegenerative disease. It is exemplified by the increasing impairment of learning and memory, although the disease may manifest itself in other ways indicating altered cognitive ability. Throughout the disease the progressive loss of neurons and synapses in the cerebral cortex leads to gross atrophy of the neural tissue. Although the cause of Alzheimer's is unknown, many believe that inflammation plays an important role and clinical studies have shown that inflammation considerably contributes to the pathogenesis of the disease.

Amyotrophic lateral sclerosis is another debilitating neurological disorder. In ALS a link between inflammation and the disease has been suggested.

In one embodiment, the neurological disorder is any disorder characterized by elevated TNF-a, and can include disorders such as stroke, depression, post-traumatic stress syndrome and traumatic brain injury.

In some embodiments, the disorders that can be treated by the scTNFR2 fusion proteins described herein include demyelinating disorders, such as but not limited to multiple sclerosis (MS), including primary progressive or relapsing-remitting MS, or optic neuritis. Other disorders such as, but not limited to, pain, which may include neuropathic pain, may be treated with the TNFR2 agonists described herein.

### Other Fusion Proteins

Because the .purpose of the Fc protein moiety in this invention is solely to increase circulating half-life, one skilled in the art will recognize that the scTNFR2 selective agonist moiety could be fused to the N-terminus of other proteins to achieve the same goal of increasing molecular size and reducing the rate of renal clearance, using the structure-activity relationships discovered in this invention. The scTNFR2 selective agonist could be fused to the N-terminus of serum albumin (Sleep. D., et al., 2013, Biochim Biophys Acta. 1830:5526-34), which both increases the hydrodynamic radius of the fusion protein relative to the TNFR2 moiety and is also recycled by the FcRN. A skilled artisan would also recognize that the scTNFR2 selective agonist moiety of this invention could also be fused to the N-terminus of recombinant non-immunogenic amino acid polymers. Two examples of non-immunogenic amino acid polymers developed for this purpose are XTEN polymers, chains of A, E, G, P. S. and T amino acids (Schellenberger, V., et. al., 2009, Nat Biotechnol. 27:1186-90)), and PAS polymers, chains of P. A, and S amino acid residues (Schlapschy, M., et. al., 2007, Protein Eng Des Scl. 20:273-84).

### Combination Treatments

Treatments that currently are available for MS include glatiramer acetate, interferonp, natalizumab, and mitoxanthrone. In general, these drugs suppress the immune system in a nonspecific fashion and only marginally limit the overall progression of disease. (Lubetzki et al. (2005), Curr. Opin. Neurol. 18:237-244). Thus, there exists a need for developing therapeutic strategies to better treat MS. As described herein, scTNFR2 find use in treating MS. These molecules find particular use when combined with currently available MS therapies as known in the art and as described herein. For instance, scTNFR2 agonists may be combined in a therapeutic regimen with glatiramer acetate, interferon-p, natalizumab, and mitoxanthron or other molecules, such as bardoxolone methyl or variants thereof.

As another example, in the treatment of Alzheimer's Disease (AD), a scTNFR2 agonist protein may be administered to an individual in combination therapy with one or more additional therapeutic agents for the treatment of AD. Suitable additional therapeutic agents include, but are not limited to, acetylcholinesterase inhibitors, including, but not limited to, Aricept (donepezil), Exelon (rivastigmine), metrifonate, and tacrine (Cognex); non-steroidal anti-inflammatory agents, including, but not limited to, ibuprofen and indomethacin; cyclooxygenase-2 (Cox2) inhibitors such as Celebrex; and monoamine oxidase inhibitors, such as Selegilene (Eldepryl or Deprenyl). Dosages for each of the above agents are known in the art. For example, Aricept is generally administered at 50 mg orally per day for 6 weeks, and, if well tolerated by the individual, at 10 mg per day thereafter.

In one embodiment, treatment of the scTNFR2 agonist in a therapeutic regimen in combination with the co-therapies as described herein results in synergistic efficacy as compared to either of the treatments alone. By “synergistic” is meant that efficacy is more than the result of additive efficacy of the two treatments alone.

In one embodiment treatment of the scTNFR2 agonist in a therapeutic regimen includes the combination of steroidal anti-inflammatory molecules, such as but not limited to dexamethasone and the like or non-steroidal anti-inflammatory molecules.

In addition, the scTNFR2 agonist may be formulated alone as a topical therapy or used in combination with or treated in a regimen with corticosteroids for treatment of autoimmune skin disorders such as psoriasis, eczema and burns (including sunburn). For instance, bath solutions and moisturizers, mineral oil and petroleum jelly which may help soothe affected skin and reduce the dryness which accompanies the build-up of skin on psoriatic plaques may be used formulated with or in a therapeutic regimen with scTNFR2 agonist as described herein. In addition, medicated creams and ointments applied directly to psoriatic plaques can help reduce inflammation, remove built-up scale, reduce skin turn over, and clear affected skin of plaques. Ointment and creams containing coal, tar, dithranol (anthralin), corticosteroids like desoximetasone (Topicort), fluocinonide, vitamin D3 analogs (for example, calcipotriol), and retinoids find use when combined with scTNFR2 agonist for topical application to the skin for treatment of autoimmune skin disorders.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the present invention.

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Generation and Characterization of TNFR2 Selective Agonist

### Example 1 Expression of TNFR2 Agonist Molecules

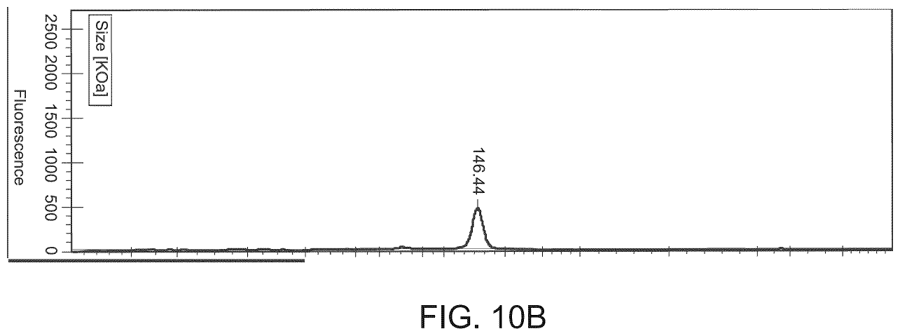

TNFR2-selective TNF variants, which are composed of a covalently stabilized human TNFR2-selective (D143N/A145R) single-chain TNF (SCTNF_{R2}) were fused to Fc dimerization domains resulting in a protein that is, with respect to TNF domains, hexameric (Fc-scTNFR2). The purity of the recombinant proteins was confirmed by SDS/PAGE and immunoblot analysis. Under reducing conditions, the TNF variants exhibited an appropriate molecular mass. Under nonreducing conditions the expected dimer was observed. The oligomerization state of Fc-scTNFR2 was further characterized by capillary electrophoresis. Fc-scTNFR2 elutes as a single major peak, indicating high purity. An exemplary electropherogram is shown in FIGS. 10A-10B for SEQ ID NO: 101.

Each gene sequence was cloned into a high expression mammalian vector. Each completed construct was sequence confirmed before proceeding to DNA scale up. Each DNA expression construct was scaled up to the appropriate amount for transfection. The plasmid DNA was run on agarose gel for quality assessment and sequence confirmed before proceeding to transfection. Suspension HEK293 cells were seeded in a shake flask and were expanded using serum-free chemically defined medium. On the day of transfection, the expanded cells were seeded into a new flask with fresh medium. Each DNA construct was transiently transfected into HEK293 cells using standard methods. The cells were maintained as a batch-fed culture until the end of the production run. The conditioned media from the transient production run was harvested and clarified by centrifugation and filtration. The supernatant was loaded over a Protein A column preequilibrated with binding buffer. Washing buffer was passed through the column until the OD280 value (NanoDrop, Thermo Scientific) was measured to be zero. The target protein was eluted with a low pH buffer, fractions were collected, and the OD280 value of each fraction was recorded. Fractions containing the target protein were pooled and filtered through a 0.2 pm membrane filter. The protein concentration was calculated from the OD280 value and the calculated extinction coefficient. CE-SDS analysis of the target protein was performed using LabChip GXII (Perkin Elmer).

### Example 2 TNFR2 Binding

TNF receptor selectivity of Fc-scTNFR2 is analyzed by binding studies with immobilized huTNFR1-Fc and huTNFR2-Fc fusion proteins. Fc-scTNFR2 does not interact with huTNFR1, but the fusion protein efficiently binds to huTNFR2. In contrast, soluble human TNF (huTNF) efficiently binds to huTNFR1, whereas it less effectively with huTNFR2.

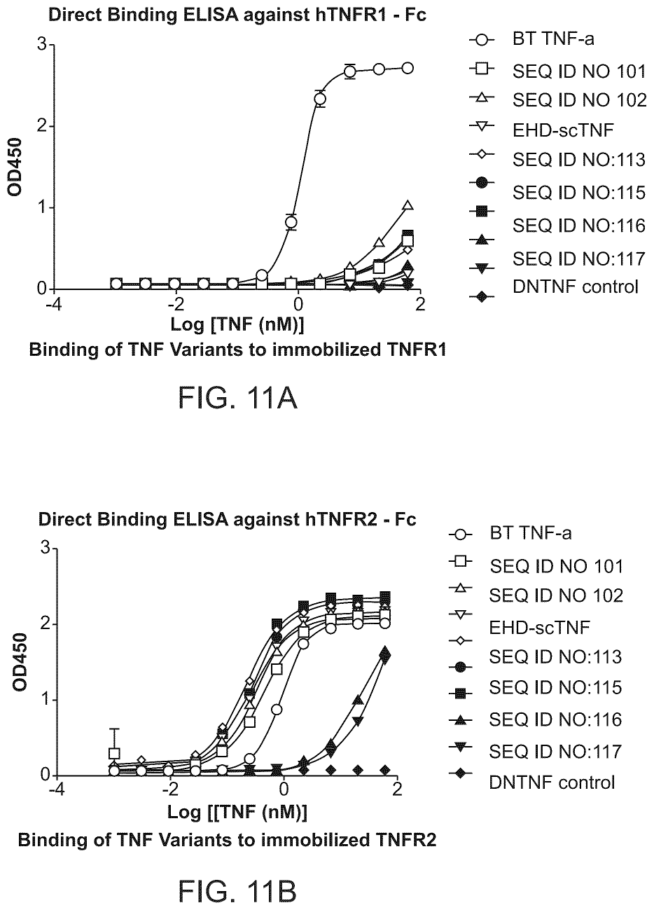

Wells were coated with 1 pg/mL hTNFR1-Fc or hTNFR2-Fc in PBS, 4° C. overnight then blocked with 3% milk in PBS, RT 1.5 hours. Primary incubation: TNF variant proteins, RT 1 hour (starting from 60 nM, 1:3 dilution). Primary detection antibody: 1 ug/mL TNF alpha monoclonal antibody (F6C5), RT 1 hour. Secondary detection antibody: HRP conjugated goat anti-mouse antibody (1:5000 dilution), RT 1 hour. Data shown in FIGS. 11A-11B. Calculated binding affinity follows:

Binding to TNFR1

| TNF Variant | Kd (nM) |
|---|---|
| TNF-a | 1.12 |
| SEQ IDNO: 101 | n/a |
| SEQ ID NO: 102 | n/a |
| EHD-scTNFr2 | n/a |
| SEQ ID NO: 113 | n/a |
| SEQ ID NO: 114 | Did not express |
| SEQ ID NO: 115 | n/a |
| SEQIDNO: 116 | n/a |
| SEQ ID NO: 117 | n/a |
| IgG4 Control | n/a |

Binding to TNFR2

| TNF Variant | Kd(nM) |
|---|---|
| TNF-a | 0.90 |
| SEQ ID NO: 101 | 0.44 |
| SEQ ID NO: 102 | 0.27 |
| EHD-scTNFr2 | 0.33 |
| SEQ ID NO: 113 | 0.21 |
| SEQ ID NO: 114 | Did not express |
| SEQ ID NO: 115 | 0.28 |
| SEQ ID NO: 116 | 0.30 |
| SEQ ID NO: 117 | n/a |
| IgG4 Control | n/a |

### Example 3 Cell Based TNFR2 Assay

Fc-scTNFR2 does not activate TNFR1-dependent cell death in L929, verifying that Fc-scTNFR2 had lost affinity for TNFR1 due to the mutations D143N/A145R. In contrast, Fc-scTNFR2 efficiently induced cell death in Kym-1 cells, which endogenously express both TNF receptors and are highly sensitive to endogenous TNF-induced TNFR1 mediated cytotoxicity. Thus, TNFR2 signaling can be measured as an increase in cell death in Kym-1 cells.

Kym-1 cells (1.5×104 cells/well) were grown in 96-well white opaque cell culture plates (Perkin Elmer) overnight. The cells were incubated with 8 concentrations of TNF muteins (100, 10, 1, 0.1, 0.01, 0.001, 0.0001 and 0.00001 ng/ml) in triplicates for 24 h at 37° C. and 5% CO2. Cell viability was analyzed at 24 h by Cell Titer Gio assay (Promega). SEQ ID NO: 114 did not express and therefore could not be tested. SEQ ID NO:117 did not induce cell death under any concentrations consistent with its inability to bind TNFR2 as shown in Example 2.

1. A fusion protein comprising:
a. a first TNF homology domain (THD) comprising D143N/A145R mutations, wherein the THD has at least 95% identity to SEQ ID NO: 3;
b. a second THD comprising D143N/A145R mutations, wherein the THD has at least 95% identity to SEQ ID NO: 3;
c. a third THD comprising D143N/A145R mutations, wherein the THD has at least 95% identity to SEQ ID NO: 3;
d. an immunoglobulin Fc domain; and
e. a first linker peptide covalently linking the first and second THDs and a second linker covalently linking the second and third THDs.

2. (canceled)

3. The fusion protein according to claim 1, wherein at least one of said THDs comprises SEQ ID NO: 3.

4. The fusion protein according to claim 3, wherein each of said THDs comprises SEQ ID NO: 3.

5. (canceled)

6. The fusion protein according to claim 5, wherein said Fc domain is covalently linked to said THD by a third peptide linker.

7. (canceled)

8. The fusion protein according to claim 6, wherein said first, second or third linker is comprised of serine or glycine amino acids.

9. (canceled)

10. The fusion protein of claim 1, wherein the immunoglobulin Fc domain is an IgG1, IgG2 or IgG4 immunoglobulin Fc domain.

11. The fusion protein of claim 10, wherein the IgG1 immunoglobulin Fc domain comprises an N297A mutation.

12. The fusion protein of claim 10, wherein the immunoglobulin Fc domain comprises is selected from the group consisting of SEQ ID NO:6, SEQ ID NO: 12 and SEQ ID NO:18.

13. (canceled)

14. The fusion protein of claim 10, wherein the fusion protein comprises SEQ ID NO: 31, SEQ ID NO:32, SEQ ID NO:34 or SEQ ID NO:35.

15. The fusion protein according to claim 10, wherein said Fc is an IgG4 Fc domain.

16. The fusion protein according to claim 15, wherein said Fc IgG4 Fc domain comprises the sequence as shown in SEQ ID NO:12.

17. The fusion protein according to claim 16, wherein said fusion protein comprises a sequence selected from the group consisting of SEQ ID NO:101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO:118, SEQ ID NO: 119 and SEQ ID NO:120.

18. The fusion protein of claim 1, wherein the fusion protein selectively activates TNFR2 over TNFR1.

19-21. (canceled)

22. A pharmaceutical composition comprising the fusion protein of claim 1.

23. A nucleic acid encoding a fusion protein according to claim 1.

24. A nucleic acid encoding the fusion protein according to claim 10.

25. The nucleic acid according to claim 24, comprising the sequence of SEQ ID NO: 36, SEQ ID NO:37, SEQ ID NO:38 or SEQ ID NO:39.

26. A cell comprising the nucleic acid according to claim 23.

27-28. (canceled)

29. A method of treating demyelinating disease in a patient in need thereof comprising administering a fusion protein according to claim 1 to said patient.

30. (canceled)

31. A method of treating pain in a patient in need thereof comprising administering a fusion protein according to claim 1 to said patient.
